(19) United States
(12) Patent Application Publication
(10) Pub. No.: US 2011/0117668 A1
(43) Pub. Date: May 19, 2011

(54) SELF-POWERED SMART DIAGNOSTIC DEVICES

(75) Inventors: Patrick S. Stayton, Seattle, WA (US); Gonzalo Jose Domingo-Villegas, Seattle, WA (US); Allison Golden, Seattle, WA (US); Jriuan Lai, Seattle, WA (US); Michael A. Nash, Seattle, WA (US); Bernhard H. Weigl, Seattle, WA (US); Nuvala Tofig Gana Fomban, Seattle, WA (US); Paul Labarre, Suquamish, WA (US)

(73) Assignee: University of Washington Through its Center for Commercialization, Seattle, WA (US)

(21) Appl. No.: 12/942,919

(22) Filed: Nov. 9, 2010

Related U.S. Application Data
(60) Provisional application No. 61/259,545, filed on Nov. 9, 2009.

(51) Int. Cl. G01N 1/18; B01D 21/00; G01N 21/75; C12M 1/34

(52) U.S. Cl. 436/178; 422/527; 422/420; 435/287.7; 435/287.2

(57) ABSTRACT

Devices and methods are provided for immobilizing a diagnostic target (e.g., indicative of a disease) from a solution (e.g., a biological fluid). The diagnostic target is first bound to a capture conjugate that includes a reversibly-associative polymer moieties attached to a first binding moiety that binds to the diagnostic target. Once the diagnostic target is bound to the capture conjugate, the solution is subjected to a change in heat and/or pH to cause the reversibly-associative polymer moieties to aggregate. The aggregates are then immobilized (e.g., via filtration).

### CROSS-REFERENCE TO RELATED APPLICATION

This application claims the benefit of U.S. Provisional Application No. 61/259,545, filed Nov. 9, 2009, which application is incorporated herein by reference in its entirety.

### STATEMENT OF GOVERNMENT LICENSE RIGHTS

This invention was made with Government support under Contract No. EB000252 awarded by the National Institutes of Health. The Government has certain rights in the invention.

### BACKGROUND

The current healthcare system has many strengths, but one of its primary vulnerabilities lies in the inequitable coverage to many economically poor, disadvantaged, and minority adult and childhood populations. These inequities are intrinsically unfair, but raise equally problematic challenges from a general public healthcare perspective. Infectious disease reservoirs and transmission sources are strongly over-represented in these populations and this connects the problem to all sectors of society. Because these populations live and move loosely or unconnected to the healthcare system, there is a key need and opportunity to first diagnose at points of intersection with outreach utilities, public institutions, and perhaps educational institutions.

Infectious diseases are sometimes diagnosed using an immunoassay, which is a biochemical test measuring the level of a substance in a biological liquid, typically using the reaction of antibodies to their recombinant antigens. Some of these assays, such as enzyme-linked immunosorbent assay (ELISA), are relatively useful for point-of-care (POC) diagnosis of infectious diseases. However, improvements in the speed, sensitivity, cost, and ease of use of immunoassays are desirable.

So as to increase reliability, and reduce the cost, of POC diagnosis of infectious diseases, a low-cost, non-instrumented (i.e., self-powered), easy-to-use device that reliably performs initial infectious disease diagnoses in low-technology environments is required.

### SUMMARY

In one aspect, a device is provided for immobilizing a diagnostic target (e.g., antibody) from a solution. In one embodiment, the device comprises: a capture surface (e.g., a membrane) configured to immobilize an aggregate from a solution comprising a biological fluid and the aggregate, wherein the aggregate comprises a plurality of capture complexes each comprising the diagnostic target bound to a first binding moiety having a temperature-responsive polymer moiety attached thereto, wherein the plurality of capture complexes are aggregated together through self-associative binding between the temperature-responsive polymer moiety on each of the capture complexes; a self-contained (e.g., non-electric, chemical) source of heat configured to deliver a predetermined amount of heat for a predetermined amount of time to the solution, wherein the predetermined amount of heat is sufficient to raise the temperature of the solution above a lower critical solution temperature of the temperature-responsive polymer for the predetermined amount of time; and fluidic-transport means configured to move the solution across the capture surface.

In another aspect, a method for concentrating a diagnostic target from a solution using a device is provided. In one embodiment, the device comprises a capture surface configured to immobilize an aggregate from a solution, a self-contained source of heat configured to deliver a predetermined amount of heat for a predetermined amount of time to the solution, and a fluidic-transport means configured to move the solution across the capture surface, wherein the solution comprises a biological fluid and a capture complex comprising the diagnostic target bound to a capture conjugate comprising a temperature-responsive polymer moiety bound to a first binding moiety (e.g., antibody) that has a binding affinity to the diagnostic target. The method for using the device includes the steps of: heating the solution with the self-contained source of heat to a temperature above a lower critical solution temperature (LCST) of the temperature-responsive polymer moiety to provide an aggregate solution comprising the biological fluid and aggregates comprising a plurality of capture complexes aggregated through self-associative binding between the temperature-responsive polymer moieties on each of the capture complexes; and flowing the aggregate solution past a capture surface configured to immobilize the aggregate, providing a captured aggregate.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1E are diagrammatic illustrations of an exemplary method for immobilizing a diagnostic target from a solution as provided herein;

FIG. 2 is a partial cross sectional isometric view of an exemplary embodiment of a device of the present invention useful for immobilizing a diagnostic target on a capture surface;

FIGS. 3A and 3B are cross sectional views of the device illustrated in FIG. 2;

FIG. 4 is a partial cross sectional isometric view of a representative device of the invention utilizing a wicking system for immobilizing a diagnostic target from a solution onto a capture surface;

FIGS. 5A through 5E are diagrammatic illustrations of an exemplary method for immobilizing a diagnostic target from a solution as provided herein using a reporting moiety;

FIGS. 6A through 6E are diagrammatic illustrations of an exemplary method for immobilizing a diagnostic target from a solution as provided herein using a magnetic moiety;

FIG. 7 is a partial cross sectional isometric view of a representative device useful for magnetically immobilizing a diagnostic target according to the present invention;

FIG. 8 illustrates the reaction scheme for conjugating a temperature-responsive polymer moiety to an antibody to form a capture conjugate as provided herein;

FIG. 9 is an SDS-PAGE gel image used to confirm the conjugation of polymer to antibody as illustrated in FIG. 8;

FIG. 10 is a graph illustrating the performance of a chemical heater as is useful as a self-contained source of heat in the present invention;

FIG. 11 is a graph illustrating the detection of p24 detected in an exemplary experiment according to the present invention;

FIG. 12 illustrates a side-by-side comparison of photographs of visual detection methods for isolating and detecting the PFHRP2 malaria antibody using the methods and devices of the invention (left column) and a commercially available rapid flow test as known in the prior art;

FIG. 13 is a graph illustrating the detection of PFHRP2 visually with machine vision for samples of two different volumes;

FIG. 14 is a diagrammatic illustration of a serology measles assay according to the present invention whereby an anti-measles IgM is detected using conjugates including a gold reporting moiety and temperature-responsive polymer moieties; and

FIG. 15 is a graph illustrating the strength of visual signal recorded using machine vision for “positive” samples having anti-measles IgM in the sample, and “negative” samples having normal human plasma undosed with IgM.

### DETAILED DESCRIPTION

The present invention provides a potentially low-cost, non-instrumented (e.g. self-powered), easy-to-use device and method useful for initial infectious disease diagnoses in low technology environments. Point-of-care (POC) devices, such as those provided herein that require no instrumentation have an intrinsic advantage in settings that are somewhat removed from mainstream healthcare: they can be stored at a health care provider's office until needed and require little training and no service or other support that is typically required for instrument-based diagnostics. The present invention combines stimuli-responsive reagents and non-instrumented detection systems to achieve non-instrumented POC diagnosis of diseases, such as, for example, HIV, malaria, and measles.

In the present invention, temperature-responsive polymers are integrated into a device having self-powered (e.g., chemical) heating, as will be described in more detail below. The combination of these two features allows for an inexpensive, non-instrumented diagnostic assay for infectious diseases.

Accordingly, in one aspect, a device is provided for immobilizing a diagnostic target (e.g., antibody) from a solution. In one embodiment, the device comprises: a capture surface (e.g., a membrane) configured to immobilize an aggregate from a solution comprising a biological fluid and the aggregate, wherein the aggregate comprises a plurality of capture complexes each comprising the diagnostic target bound to a first binding moiety having a temperature-responsive polymer moiety attached thereto, wherein the plurality of capture complexes are aggregated together through self-associative binding between the temperature-responsive polymer moiety on each of the capture complexes; a self-contained (e.g., non-electric, chemical) source of heat configured to deliver a predetermined amount of heat for a predetermined amount of time to the solution, wherein the predetermined amount of heat is sufficient to raise the temperature of the solution above a lower critical solution temperature of the temperature-responsive polymer for the predetermined amount of time; and fluidic-transport means configured to move the solution across the capture surface.

In another aspect, a method for concentrating a diagnostic target from a solution using a device is provided. In one embodiment, the device comprises a capture surface configured to immobilize an aggregate from a solution, a self-contained source of heat configured to deliver a predetermined amount of heat for a predetermined amount of time to the solution, and a fluidic-transport means configured to move the solution across the capture surface, wherein the solution comprises a biological fluid and a capture complex comprising the diagnostic target bound to a capture conjugate comprising a temperature-responsive polymer moiety bound to a first binding moiety (e.g., antibody) that has a binding affinity to the diagnostic target. The method for using the device includes the steps of: heating the solution with the self-contained source of heat to a temperature above a lower critical solution temperature (LCST) of the temperature-responsive polymer moiety to provide an aggregate solution comprising the biological fluid and aggregates comprising a plurality of capture complexes aggregated through self-associative binding between the temperature-responsive polymer moieties on each of the capture complexes; and flowing the aggregate solution past a capture surface configured to immobilize the aggregate, providing a captured aggregate.

As will be described in more detail below, a central feature of the present invention is the use of “stimuli-responsive polymers”. As used herein, the term “stimuli-responsive polymers” refers to a general class of polymers (or polymer moieties) that exhibit a change from a hydrophobic state to a hydrophilic state as the result of an environmental stimulus. Two representative stimuli-responsive polymers useful in the present invention are temperature-responsive polymers and pH-responsive polymers. As used herein, the term “temperature-responsive polymer” refers to polymers that are reversibly self-associative in response to temperature. Particularly, above a lower critical solution temperature (LCST), temperature-responsive polymers are self-associative, meaning the polymers bind to themselves and other similar temperature-responsive polymers. Below the LCST, the polymer is hydrophilic and highly solvated, while above the LCST, it is aggregated and phase separated. Of use in the present invention is the sharp transition from individual chains to the aggregated state over a very narrow temperature range of a few degrees. The change is completely reversible, and reversal of the stimulus results in the polymer going back into solution rapidly.

Similarly, pH-responsive polymers transition from hydrophobic to hydrophilic based on a critical pH. PH-responsive polymers are known to those of skill in the art, and are described in the context of affinity binding in U.S. Pat. No. 7,625,764, incorporated herein by reference in its entirety. Representative pH-responsive polymers include polymers formed from monomers that include acrylic acid, methacrylic acid, propyl acrylic acid, butyl acrylate, butyl methacrylate, and alkyl-substituted acrylic acids in general.

Other responsive polymers are known to those of skill in the art, for example light-sensitive polymers. Any polymer capable of forming aggregates, as disclosed herein, are useful in the present invention.

The present invention is primarily disclosed in terms of temperature-responsive polymers. However, it will be appreciated by those of skill in the art that pH-responsive polymers can be substituted for temperature-responsive polymers in the methods and devices disclosed herein.

Additionally, some polymers are both temperature- and pH-responsive. Therefore, certain methods and devices of the invention include the use of both temperature and pH to aggregate polymers.

Temperature-responsive polymers are known to those of skill in the art, with the most common being poly(N-isopropylacrylamide) (PNIPAAm). Other temperature-responsive polymers include those formed from monomers including tert-butyl methacrylate, tert-butyl acrylate, butyl methacrylate, butylacrylate, dimethylaminoethyl acrylamide, and propylacrylic acid.

As set forth in U.S. Pat. No. 7,625,764, incorporated herein by reference in its entirety, temperature-responsive polymers can be used to bind two or more distinct objects (e.g., particles, molecules, etc.) through the self-associative interaction of temperature-responsive polymer moieties attached to each object in a solution above the LCST.

The presence of the stimuli-responsive polymer moiety on a conjugate provides for the formation of the aggregate on the application of an appropriate stimulus. For example, when the conjugates bear a thermally-responsive polymer, the aggregate is formed by heating the liquid to a temperature above the lower critical solution temperature of the thermally-responsive polymer (e.g., a polymer comprising N-isopropylacrylamide repeating units, an N-isopropylacrylamide polymer or copolymer). When the conjugates bear a pH-responsive polymer, the aggregate is formed by adjusting the pH of the liquid to a pH that causes the polymers to become associative (e.g., a polymer comprising acrylic acid or alkylacrylic acid repeating units, an acrylic acid or alkylacrylic acid polymer or copolymer). A representative pH-responsive polymer is an N-isopropylacrylamide/methylacrylic acid/tert-butyl methacrylate copolymer such as poly(N-isopropylacrylamide-co-methylacrylic acid-co-tert-butyl methacrylate. When the conjugates bear an ionic strength-responsive polymer, the co-aggregate is formed by adjusting the ionic strength of the liquid such that the polymers become associative. Similarly, when the conjugates bear a light-responsive polymer, the co-aggregate is formed by irradiating the liquid with a wavelength of light effective to cause the polymers to become associative.

The stimuli-responsive polymer can be any polymer having a stimuli-responsive property. The stimuli-responsive polymer can be any one of a variety of polymers that change their associative properties (e.g., change from hydrophilic to hydrophobic) in response to a stimulus. The stimuli-responsive polymer responds to changes in external stimuli such as the temperature, pH, light, photo-irradiation, exposure to an electric field, ionic strength, and the concentration of certain chemicals by exhibiting property change. For example, a thermally-responsive polymer is responsive to changes in temperature by exhibiting a LCST in aqueous solution. The stimuli-responsive polymer can be a multi-responsive polymer, where the polymer exhibits property change in response to combined simultaneous or sequential changes in two or more external stimuli.

The stimuli-responsive polymers may be synthetic or natural polymers that exhibit reversible conformational or physico-chemical changes such as folding/unfolding transitions, reversible precipitation behavior, or other conformational changes to in response to stimuli, such as to changes in temperature, light, pH, ions, or pressure. Representative stimuli-responsive polymers include temperature-sensitive polymers (also referred to herein as “temperature-responsive polymers” or “thermally-responsive polymers”), pH-sensitive polymers (also referred to herein as “pH-responsive polymers”), and light-sensitive polymers (also referred to herein as “light-responsive polymers”).

Stimulus-responsive polymers useful in making the particles described herein can be any which are sensitive to a stimulus that causes significant conformational changes in the polymer. Illustrative polymers described herein include temperature-, pH-, ion- and/or light-sensitive polymers. Hoffman, A. S., “Intelligent Polymers in Medicine and Biotechnology”, Artif. Organs. 19:458-467, 1995; Chen, G. H. and A. S. Hoffman, “A New Temperature- and Ph-Responsive Copolymer for Possible Use in Protein Conjugation”, Macromol. Chem. Phys. 196:1251-1259. 1995; Irie, M. and D. Kungwatchakun, “Photoresponsive Polymers. Mechanochemistry of Polyacrylamide Gels Having Triphenylmethane Leuco Derivatives”, Makromol. Chem., Rapid Commun. 5:829-832, 1985; and Irie, M., “Light-induced Reversible Conformational Changes of Polymers in Solution and Gel Phase”, ACS Polym. Preprints, 27(2):342-343, 1986; which are incorporated by reference herein.

Stimuli-responsive oligomers and polymers useful in the particles described herein can be synthesized that range in molecular weight from about 1,000 to 30,000 Daltons. In one embodiment, these syntheses are based on the chain transfer-initiated free radical polymerization of vinyl-type monomers, as described herein, and by (1) Tanaka, T., “Gels”, Sci. Amer. 244:124-138. 1981; (2) Osada, Y. and S. B. Ross-Murphy, “Intelligent Gels”, Sci. Amer, 268:82-87, 1993; (3) Hoffman, A. S., “Intelligent Polymers in Medicine and Biotechnology”, Artif. Organs 19:458-467, 1995; also Macromol. Symp. 98:645-664, 1995; (4) Feijen, J., et al., “Thermosensitive Polymers and Hydrogels Based on N-isopropylacrylamide”, 11th European Conf. on Biomtls:256-260, 1994; (5) Monji, N. and A. S. Hoffman, “A Novel Immunoassay System and Bioseparation Process Based on Thermal Phase Separating Polymers”, Appl. Biochem. and Biotech. 14:107-120, 1987; (6) Fujimura, M., T. Mori and T. Tosa, “Preparation and Properties of Soluble-Insoluble Immobilized Proteases”, Biotech. Bioeng. 29:747-752, 1987; (7) Nguyen, A. L. and J. H. T. Luong, “Synthesis and Applications of Water-Soluble Reactive Polymers for Purification and Immobilization of Biomolecules”, Biotech. Bioeng. 34:1186-1190, 1989; (8) Taniguchi, M., et al., “Properties of a Reversible Soluble-Insoluble Cellulase and Its Application to Repeated Hydrolysis of Crystalline Cellulose”, Biotech. Bioeng. 34:1092-1097, 1989; (9) Monji, N., et al., “Application of a Thermally-Reversible Polymer-Antibody Conjugate in a Novel Membrane-Based Immunoassay”, Biochem. and Biophys. Res. Comm. 172:652-660, 1990; (10) Monji, N. C. A. Cole, and A. S. Hoffman, “Activated, N-Substituted Acrylamide Polymers for Antibody Coupling: Application to a Novel Membrane-Based Immunoassay”, J. Biomtls. Sci. Polymer Ed. 5:407-420, 1994; (11) Chen, J. P. and A. S. Hoffman, “Polymer-Protein Conjugates: Affinity Precipitation of Human IgG by Poly(N-Isopropyl Acrylamide)-Protein A Conjugates”, Biomtls. 11:631-634, 1990; (12) Park, T. G. and A. S. Hoffman, “Synthesis and Characterization of a Soluble, Temperature-Sensitive Polymer-Conjugated Enzyme, J. Biomtls. Sci. Polymer Ed. 4:493-504, 1993; (13) Chen, G. H., and A. S. Hoffman, Preparation and Properties of Thermo-Reversible, Phase-Separating Enzyme-Oligo(NIPAAm) Conjugates”, Bioconj. Chem. 4:509-514, 1993; (14) Ding, Z. L., et al., “Synthesis and Purification of Thermally-Sensitive Oligomer-Enzyme Conjugates of Poly(NIPAAm)-Trypsin”, Bioconj. Chem. 7: 121-125, 1995; (15) Chen, G. H. and A. S. Hoffman, “A New Temperature- and pH-Responsive Copolymer for Possible Use in Protein Conjugation”, Macromol. Chem. Phys. 196:1251-1259, 1995; (16) Takei, Y. G., et al., “Temperature-responsive Bioconjugates. 1. Synthesis of Temperature-Responsive Oligomers with Reactive End Groups and their Coupling to Biomolecules”, Bioconj. Chem. 4:42-46, 1993; (17) Takei, Y. G., et al., “Temperature-responsive Bioconjugates. 2. Molecular Design for Temperature-modulated Bioseparations”, Bioconj. Chem. 4:341-346, 1993; (18) Takei, Y. G., et al., “Temperature-responsive Bioconjugates. 3. Antibody-Poly(N-isopropylacrylamide) Conjugates for Temperature-Modulated Precipitations and Affinity Bioseparations”, Bioconj. Chem. 5:577-582, 1994; (19) Matsukata, M., et al., “Temperature Modulated Solubility-Activity Alterations for Poly(N-Isopropylacrylamide)-Lipase Conjugates”, J. Biochem. 116:682-686, 1994; (20) Chilkoti, A., et al., “Site-Specific Conjugation of a Temperature-Sensitive Polymer to a Genetically-Engineered Protein”, Bioconj. Chem. 5:504-507, 1994; and (21) Stayton, P. S., et al., “Control of Protein-Ligand Recognition Using a Stimuli-Responsive Polymer”, Nature 378:472-474, 1995.

The stimuli-responsive polymers useful herein include homopolymers and copolymers having stimuli-responsive behavior. Other suitable stimuli-responsive polymers include block and graft copolymers having one or more stimuli-responsive polymer components. A suitable stimuli-responsive block copolymer may include, for example, a temperature-sensitive polymer block, or a pH-sensitive block. A suitable stimuli-responsive graft copolymer may include, for example, a pH-sensitive polymer backbone and pendant temperature-sensitive polymer components, or a temperature-sensitive polymer backbone and pendant pH-sensitive polymer components.

The stimuli-responsive polymer can include a polymer having a balance of hydrophilic and hydrophobic groups, such as polymers and copolymers of N-isopropylacrylamide. An appropriate hydrophilic/hydrophobic balance in a smart vinyl type polymer is achieved, for example, with a pendant hydrophobic group of about 2-6 carbons that hydrophobically bond with water, and a pendant polar group such as an amide, acid, amine, or hydroxyl group that H-bond with water. Other polar groups include sulfonate, sulfate, phosphate and ammonium ionic groups. Preferred embodiments are for 3-4 carbons (e.g., propyl, isopropyl, n-butyl, isobutyl, and t-butyl) combined with an amide group (e.g. PNIPAAm), or 2-4 carbons (e.g., ethyl, propyl, isopropyl, n-butyl, isobutyl, and t-butyl) combined with a carboxylic acid group (e.g., PPAA). There is also a family of smart A-B-A (also A-B-C) block copolymers of polyethers, such as PLURONIC polymers having compositions of PEO-PPO-PEO, or polyester-ether compositions such as PLGA-PEG-PLGA. In one embodiment, the stimuli-responsive polymer is a temperature responsive polymer, poly(N-isopropylacrylamide) (PNIPAAm).

The stimuli-responsive polymer useful in the invention can be a smart polymer having different or multiple stimuli responsivities, such as homopolymers responsive to pH or light. Block, graft, or random copolymers with dual sensitivities, such as pH and temperature, light and temperature, or pH and light, may also be used.

Illustrative embodiments of the many different types of thermally-responsive polymers that may be conjugated to interactive molecules are polymers and copolymers of N-isopropyl acrylamide (NIPAAm). PolyNIPAAm is a thermally-responsive polymer that precipitates out of water at 32° C., which is its lower critical solution temperature (LCST), or cloud point (Heskins and Guillet, J. Macromol. Sci.-Chem. A2:1441-1455, 1968). When polyNIPAAm is copolymerized with more hydrophilic comonomers such as acrylamide, the LCST is raised. The opposite occurs when it is copolymerized with more hydrophobic comonomers, such as N-t-butyl acrylamide. Copolymers of NIPAAm with more hydrophilic monomers, such as AAm, have a higher LCST, and a broader temperature range of precipitation, while copolymers with more hydrophobic monomers, such as N-t-butyl acrylamide, have a lower LCST and usually are more likely to retain the sharp transition characteristic of PNIPAAm (Taylor and Cerankowski, J. Polymer Sci. 13:2551-2570, 1975; Priest et al., ACS Symposium Series 350:255-264, 1987; and Heskins and Guillet, J. Macromol. Sci.-Chem. A2:1441-1455, 1968, the disclosures of which are incorporated herein). Copolymers can be produced having higher or lower LCSTs and a broader temperature range of precipitation.

Thermally-responsive oligopeptides also may be incorporated into the conjugates.

Synthetic pH-responsive polymers useful in making the conjugates described herein are typically based on pH-sensitive vinyl monomers, such as acrylic acid (AAc), methacrylic acid (MAAc) and other alkyl-substituted acrylic acids such as ethylacrylic acid (EAAc), propylacrylic acid (PAAc), and butylacrylic acid (BAAc), maleic anhydride (MAnh), maleic acid (MAc), AMPS (2-acrylamido-2-methyl-1-propanesulfonic acid), N-vinyl formamide (NVA), N-vinyl acetamide (NVA) (the last two may be hydrolyzed to polyvinylamine after polymerization), aminoethyl methacrylate (AEMA), phosphoryl ethyl acrylate (PEA) or methacrylate (PEMA). pH-Responsive polymers may also be synthesized as polypeptides from amino acids (e.g., polylysine or polyglutamic acid) or derived from naturally-occurring polymers such as proteins (e.g., lysozyme, albumin, casein), or polysaccharides (e.g., alginic acid, hyaluronic acid, carrageenan, chitosan, carboxymethyl cellulose) or nucleic acids, such as DNA. pH-Responsive polymers usually contain pendant pH-sensitive groups such as —OPO(OH)2, —COOH, or —NH2 groups. With pH-responsive polymers, small changes in pH can stimulate phase-separation, similar to the effect of temperature on solutions of PNIPAAm (Fujimura et al. Biotech. Bioeng. 29:747-752 (1987)). By randomly copolymerizing a thermally-sensitive NIPAAm with a small amount (e.g., less than 10 mole percent) of a pH-sensitive comonomer such as AAc, a copolymer will display both temperature and pH sensitivity. Its LCST will be almost unaffected, sometimes even lowered a few degrees, at pHs where the comonomer is not ionized, but it will be dramatically raised if the pH-sensitive groups are ionized. When the pH-sensitive monomer is present in a higher content, the LCST response of the temperature-sensitive component may be “eliminated” (e.g., no phase separation seen up to and above 100° C.).

Graft and block copolymers of pH and temperature-sensitive monomers can be synthesized that retain both pH and temperature transitions independently. Chen, G. H., and A. S. Hoffman, Nature 373:49-52, 1995. For example, a block copolymer having a pH-sensitive block (polyacrylic acid) and a temperature-sensitive block (PNIPAAm) can be useful in the invention.

Light-responsive polymers usually contain chromophoric groups pendant to or along the main chain of the polymer and, when exposed to an appropriate wavelength of light, can be isomerized from the trans to the cis form, which is dipolar and more hydrophilic and can cause reversible polymer conformational changes. Other light sensitive compounds can also be converted by light stimulation from a relatively non-polar hydrophobic, non-ionized state to a hydrophilic, ionic state.

In the case of pendant light-sensitive group polymers, the light-sensitive dye, such as aromatic azo compounds or stilbene derivatives, may be conjugated to a reactive monomer (an exception is a dye such as chlorophyllin, which already has a vinyl group) and then homopolymerized or copolymerized with other conventional monomers, or copolymerized with temperature-sensitive or pH-sensitive monomers using the chain transfer polymerization as described above. The light sensitive group may also be conjugated to one end of a different (e.g., temperature) responsive polymer. A number of protocols for such dye-conjugated monomer syntheses are known.

Although both pendant and main chain light sensitive polymers may be synthesized and are useful for the methods and applications described herein, the preferred light-sensitive polymers and copolymers thereof are typically synthesized from vinyl monomers that contain light-sensitive pendant groups. Copolymers of these types of monomers are prepared with “normal” water-soluble comonomers such as acrylamide, and also with temperature- or pH-sensitive comonomers such as NIPAAm or AAc.

Light-sensitive compounds may be dye molecules that isomerize or become ionized when they absorb certain wavelengths of light, converting them from hydrophobic to hydrophilic conformations, or they may be other dye molecules which give off heat when they absorb certain wavelengths of light. In the former case, the isomerization alone can cause chain expansion or collapse, while in the latter case the polymer will precipitate only if it is also temperature-sensitive.

Light-responsive polymers usually contain chromophoric groups pendant to the main chain of the polymer. Typical chromophoric groups that have been used are the aromatic diazo dyes (Ciardelli, Biopolymers 23:1423-1437, 1984; Kungwatchakun and Irie, Makromol. Chem., Rapid Commun. 9:243-246, 1988; Lohmann and Petrak, CRC Crit. Rev. Therap. Drug Carrier Systems 5:263, 1989; Mamada et al., Macromolecules 23:1517, 1990, each of which is incorporated herein by reference). When this type of dye is exposed to 350-410 nm UV light, the trans form of the aromatic diazo dye, which is more hydrophobic, is isomerized to the cis form, which is dipolar and more hydrophilic, and this can cause polymer conformational changes, causing a turbid polymer solution to clear, depending on the degree of dye-conjugation to the backbone and the water solubility of the main unit of the backbone. Exposure to about 750 nm visible light will reverse the phenomenon. Such light-sensitive dyes may also be incorporated along the main chain of the backbone, such that the conformational changes due to light-induced isomerization of the dye will cause polymer chain conformational changes. Conversion of the pendant dye to a hydrophilic or hydrophobic state can also cause individual chains to expand or contract their conformations. When the polymer main chain contains light sensitive groups (e.g., azo benzene dye) the light-stimulated state may actually contract and become more hydrophilic upon light-induced isomerization. The light-sensitive polymers can include polymers having pendant or backbone azobenzene groups.

Polysaccharides, such as carrageenan, that change their conformation, for example, from a random to an ordered conformation, as a function of exposure to specific ions, such as potassium or calcium, can also be used as the stimulus-responsive polymers. In another example, a solution of sodium alginate may be gelled by exposure to calcium. Other specific ion-sensitive polymers include polymers with pendant ion chelating groups, such as histidine or EDTA.

Polymers that are responsive to changes in ionic strength can also be used.

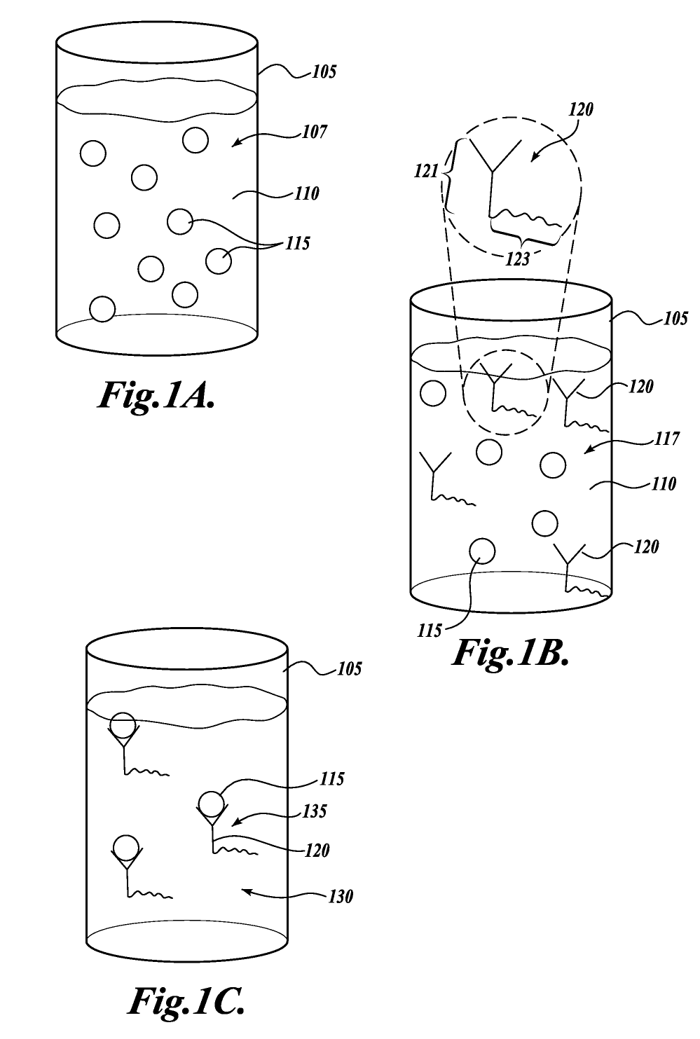

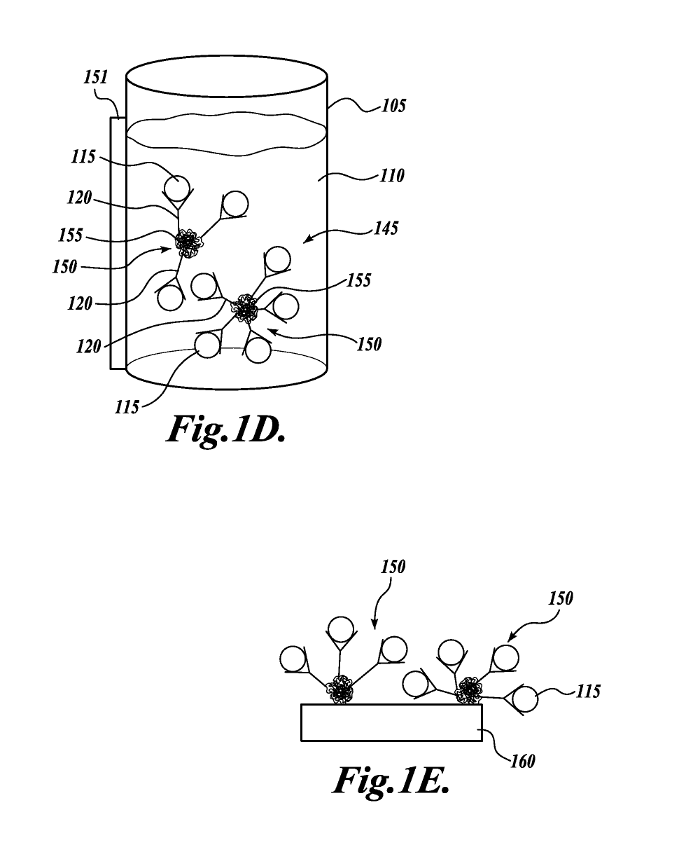

The present invention utilizes the aggregation of stimuli-responsive polymers to isolate the diagnostic target from a solution. FIGS. 1A through 1E illustrate the aggregation and capture of aggregates comprising a diagnostic target 115 from a solution 107 comprising the diagnostic target 115 and a biological fluid 110, according to the present invention.

Referring to FIG. 1A, a container 105 is illustrated holding a solution 107 comprising a biological fluid 110 and a diagnostic target 115. It will be appreciated that a container 105 is not necessary for performing the methods or devices of the present invention, although a container 105 is useful for preparing the solution 107 for processing using the present invention.

The solution 107 comprises a biological fluid 110 and a diagnostic target 115. The biological fluid 110 can be any fluid produced by an organism. Representative biological fluids are mammalian biological fluids, such as, for example, blood, mucus, urine, tissue, sputum, saliva, feces, a nasal swab, and nasopharyngeal washes.

The diagnostic target 115 is an analyte in the biological fluid 110 indicative of the presence of a disease. Representative diseases include infectious diseases such as human immunodeficiency virus (HIV), malaria, dengue, salmonella, rickettsia, influenza, chlamydia, prostate cancer and measles. In a representative embodiment, the infectious disease is present in a human being, and the presence of the infectious disease within the human being's body produces antibodies, antigens, or other biological markers that indicate the presence of the infectious disease in the body. Any of these analytes (antibodies, antigens, or other biological markers) are diagnostic targets useful in the present invention. Representative diagnostic targets include a p24 protein of human immunodeficiency virus, a PfHRP2 antigen of malaria, an aldolase antigen of malaria, NS1 antigen of dengue, flagella/somatic/Vi antigens of salmonella, nucleoprotein/hemagglutinin antigens of influenza, LPS antigen of Chlamydia, prostate-specific antigen of prostate cancer, and antibodies of diseases selected from the group including dengue, salmonella, and rickettsia

One of the central issues addressed by the present invention is the inexpensive, point-of-care, diagnosis of infectious diseases using a self-contained (self-powered) device capable of operation by untrained individuals. The present invention addresses this issue by forming aggregates 150 that include the diagnostic target 115. The aggregates 150 are formed using self-contained heat and then the aggregates 150 are immobilized for identification.

Before forming aggregates, the diagnostic target 115 is bound to a capture conjugate 120. With reference to FIG. 1B, the diagnostic targets 115 in the biological fluid 110 are combined in the solution 117 with capture conjugates 120, each of which comprise a first binding moiety 121 and a temperature-responsive polymer moiety 123. While several embodiments described herein incorporate temperature-responsive stimuli-responsive polymers, it will be appreciated that other types of stimuli-responsive polymers (e.g., pH-responsive) can also be used, or combinations of two or more types of stimuli-responsive polymers (e.g., temperature- and pH-responsive polymers).

The capture conjugates 120 bind (e.g., spontaneously) to the diagnostic targets 115, as illustrated in FIG. 1C, to form capture complexes 135.

The first binding moiety 121 is, therefore, defined as a moiety having a binding affinity to the diagnostic target 115. Depending on the composition of the diagnostic target 115, the first binding moiety 121 may be an antibody, an antigen, or other chemical functional group having a binding affinity to the diagnostic target 151.

The first binding moiety 121 can also be part of a serology system whereby the capture conjugate 120 may comprise three or more moieties to provide binding to an anti-[disease] antibody, or the like. In such an embodiment, the capture conjugate 120 comprises the temperature-responsive polymer moiety 123, and a first binding moiety 121 comprising an anti-[disease] antigen antibody bound to a disease antigen via the antibody. The antigen on the first binding moiety 121 then provides binding to the anti-[disease] antibody, which is the diagnostic target 115.

The temperature-responsive polymer moiety 123 is bound to the first binding moiety 121 so as to form the capture conjugate 120. The temperature-responsive polymer moiety is self-associative in response to temperature change greater than the LCST, as has been described previously. Representative temperature-responsive polymer moieties are PNIPAAm moieties.

The capture conjugate 120 (and further conjugates, such as the reporting conjugate and the magnetic particles described below) can be in a dried form and added to the biological fluid 110 or solvated in a solution added to the biological fluid 110. One advantage of the use of dried capture conjugate 120 is to avoid the need for refrigeration of a solution containing solvated capture conjugate 120.

Aggregates 150 of the capture complex 135 are formed, with reference to FIG. 1D, by providing the capture complexes 135 in a solution 145 heated above the LCST of the temperature-responsive polymer moieties 123 on each of the capture conjugates 120. This rise in temperature above the LCST causes the temperature-responsive polymer moieties 123 to become self-associative so as to form aggregates 150 comprising a plurality of capture complexes 135 bound together through the associative binding 155 between temperature-responsive polymer moieties 123 on each of the capture complexes 135.

In the embodiment illustrated in FIG. 1D, a heater 151 provides heat to the solution 145 so as to raise the temperature of the solution above the LCST and provide the aggregates 150. The aggregates 150 are of a size significantly larger than that of the diagnostic target 115.

In the present invention, the immobilization of the diagnostic target 115 is accomplished in one embodiment by first aggregating the aggregates 150. The aggregates 150 are then pushed through a membrane (e.g., filter) having a surface chemistry that adheres the aggregates 150 to membrane 160 upon contact. As illustrated in FIG. 1E, the membrane 160 collects the aggregates 150 from solution as the solution 145 is passed through the filter 160. The aggregates 150 are immobilized on the surface of the membrane 160.

Regarding immobilization of the aggregates 150 on the membrane 160, any mechanism for immobilization can be implemented in the present invention. Particularly useful are chemical adhesion means. Representative chemical adhesion means include hydrogen bonding between at least one moiety on the aggregate 150 and the membrane 160; and hydrophobic-hydrophobic (or hydrophilic-hydrophilic) affinity binding. Affinity binding can be between the aggregate 150 and an untreated membrane (e.g., hydroxylated nylon) or a membrane having temperature-responsive moieties attached thereto.

After immobilization, the aggregates 150 can be further processed to identify the diagnostic targets 115 using methods known to those of skill in the art. For example, the aggregates 150 can be washed with a solution, or series of solutions, containing the reagents to perform visual indication of the presence of the diagnostic target 115, such as an enzyme-based visual indicator or using a gold particle-based visual indicator know to those of skill in the art. Alternatively, the immobilized aggregates 150 can be re-solvated in a relatively small amount of solvent and tested by lateral flow or other techniques known to those of skill in the art.

The self-contained, or self-powered, heater of the invention provides heat in certain embodiments through suitable reactions for exothermic heating. In one embodiment, the self-contained heater is not electric. In another embodiment, the self-contained heater is a chemical heater.

In the present invention, phase-change materials (PCM), such as sodium acetate trihydrate (“sodium acetate”) and parafins, can be used to stabilize a heat mixture at a defined temperature (±3° C.) independent of ambient temperatures. A PCM is a substance with a high heat of fusion which, melting and solidifying at a certain temperature, is capable of storing and releasing large amounts of energy. Heat is absorbed or released when the material changes from solid to liquid and vice versa.

The PCM can either be added to exothermic reactants or transmit heat from exothermic component to the sample. When the melting temperature of the PCM is reached, the temperature remains constant until the phase change of the entire sample completes from solid to liquid, even though the exothermic reaction may be at significantly higher temperatures. Conversely, as the exothermic reactants are used up and they cool below the PCM melting temperature, the PCM will still provide heat to the sample at the desired melting temperature until the phase change is complete.

In exemplary embodiments, saturated (or supersaturated) sodium acetate in water solution is packaged in a tri-laminate foil pouch that maintains the solution in a clean, stable environment and also prevents evaporative losses. Crystallization and heat formation are initiated by cutting into the pouch or by using an embedded metal “button” as known to those of skill in the art. Because these pouches are flexible they can be integrated into the devices of the invention in a variety of geometric configurations.

Representative ratios of sodium acetate to water (weight/weight) are from about 15% to about 30%. The ratio of sodium acetate determines the maximum temperature the solution achieves, with a smaller amount of sodium acetate resulting in higher temperature. For example, sodium acetate solutions in water of (wt/wt) 15%, 20%, 25%, and 30% yield maximum temperatures of 50° C., 46° C., 41° C., and 38° C., respectively.

PCMs are generally known in the art. For example, paraffin as a PCM is disclosed in U.S. Pat. No. 4,249,592, incorporated herein by reference in its entirety. And U.S. Pat. No. 4,332,690, incorporated herein by reference in its entirety, discloses a variety of PCMs from guest/host systems.

Besides PCMs, representative self-contained heating materials include: using evaporation of acetone (or other solvents) as an endothermic process to cool; and the use of exothermic dissolution of concentrated sulfuric acid in water.

A preferred PCM material is supersaturated sodium acetate trihydrate, which has the advantage of exhibiting constant temperature properties while also releasing heat transitioning from a stable liquid state to a crystalline structure. In this regard, sodium acetate (and similar salts) is both a chemical heat source and a PCM. As the liquid salt mixture returns to the crystalline state, it can provide the energy required by a diagnostic platform at a constant temperature. These stable mixtures can be triggered with a nucleating agent to spontaneously crystallize and release heat. The nucleating agent is often provided by a small metal concave disc that is flexed to begin the crystallization and release of stored energy as heat.

A sodium acetate heat source does not require the use of external power or batteries, thus resulting in lower waste. Sodium acetate can be recycled and reused numerous times by applying heat and converting the salt mixture from a crystalline back to a liquid state.

### Flow-Through Syringe Device

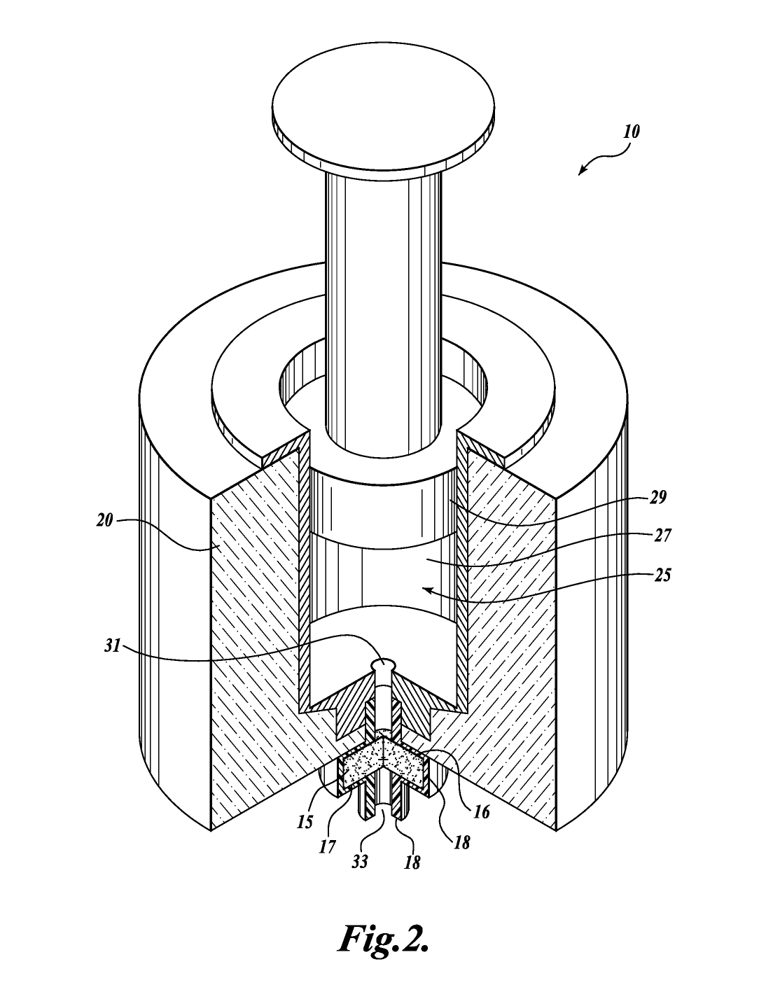

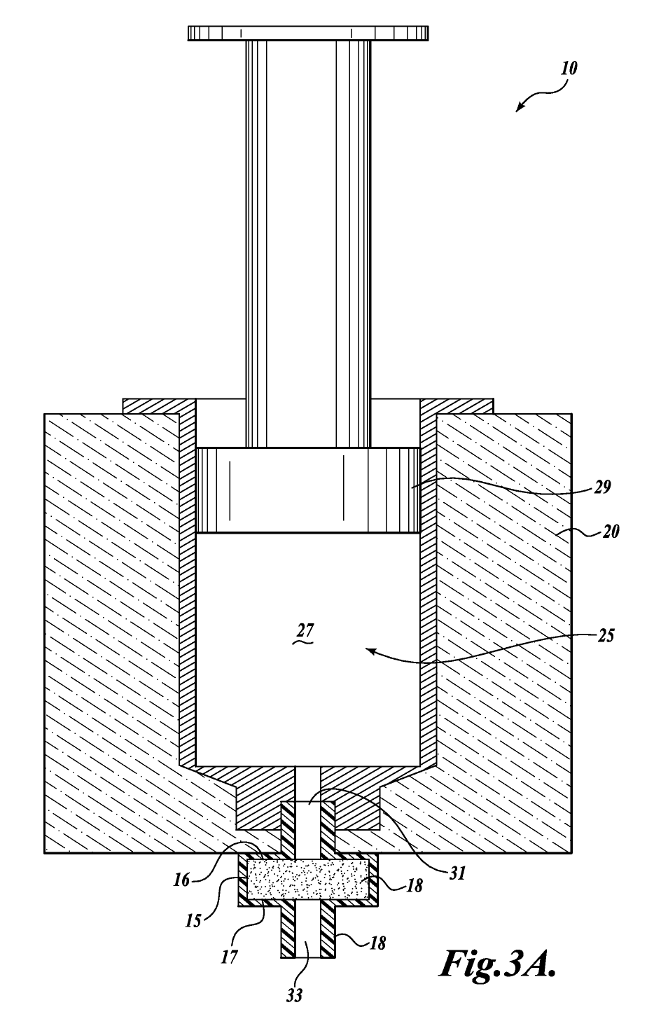

In certain embodiments of the invention, a syringe-style device is provided. The syringe provides a means for flow of a solution past a membrane for immobilizing aggregates from a solution, each aggregate containing one or more diagnostic targets (e.g., 115/215/315). Referring to FIG. 2, a syringe flow-through device 10 is illustrated in partial cross sectional isometric view. The device 10 includes a capture surface 15 (illustrated as a membrane 15). The membrane 15 is in fluid communication with a container 27 in contact with a self-contained heater 20. The container 27 is part of a syringe system 25 that comprises the container and a plunger 29 actuatable by a user or machine to increase or decrease the volume of the container 27. The container 27 is in fluid communication with the membrane 15 through a syringe outlet 31 connectively coupled to a membrane housing 18 comprising the membrane 15. The membrane 15 has an inlet surface 16 and an outlet surface 17. A fluid pushed through the syringe system 25 will travel from the container 27, through the syringe outlet 31, into contact with the inlet surface 16 of the membrane 15, through the body of the membrane 15, out of the membrane 15 through the outlet surface 17, and finally pass out of the device through the device outlet 33.

The plunger 29 acts to apply pressure on the contents of the container 27 so as to provide fluidic transport within the syringe system 25. Therefore, the syringe system 25 described herein is a representative example of a fluidic-transport means configured to move a solution across a capture surface.

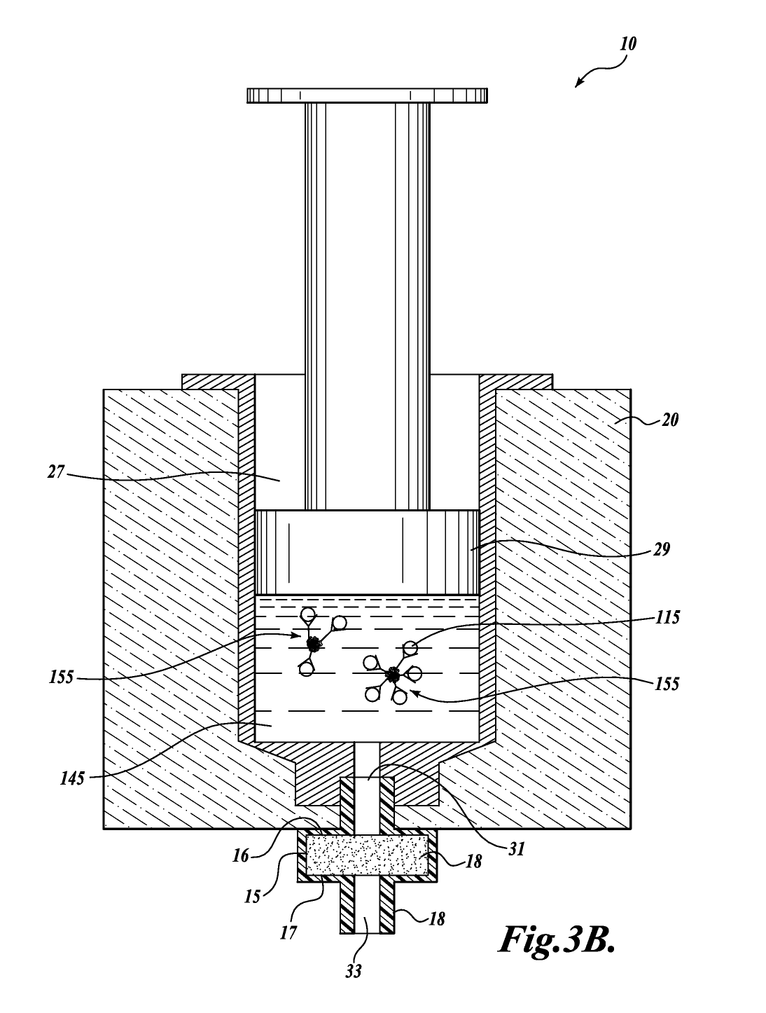

FIG. 3A is a cross sectional view of the device 10 illustrated in FIG. 2.

FIG. 3B is another cross sectional view of the device of FIG. 2. FIG. 3B includes a solution 145 comprising aggregates 150 (as described above with reference to FIGS. 1A through 1E) in the container 27. The plunger 29 of the device 10 is in intimate contact with the solution 145, and further actuation of the plunger 29 toward the membrane 15 will drive the solution 145 and the aggregates 150 therein through the membrane 15. The aggregates 155 will be immobilized on the membrane 15.

As discussed elsewhere herein, visual or other identification techniques can be used to identify the diagnostic targets 115 on the aggregates 150 so as to provide a simple, positive indication of the presence of the diagnostic target 115 in the solution 145.

### Flow-Through Absorbent Pad Device

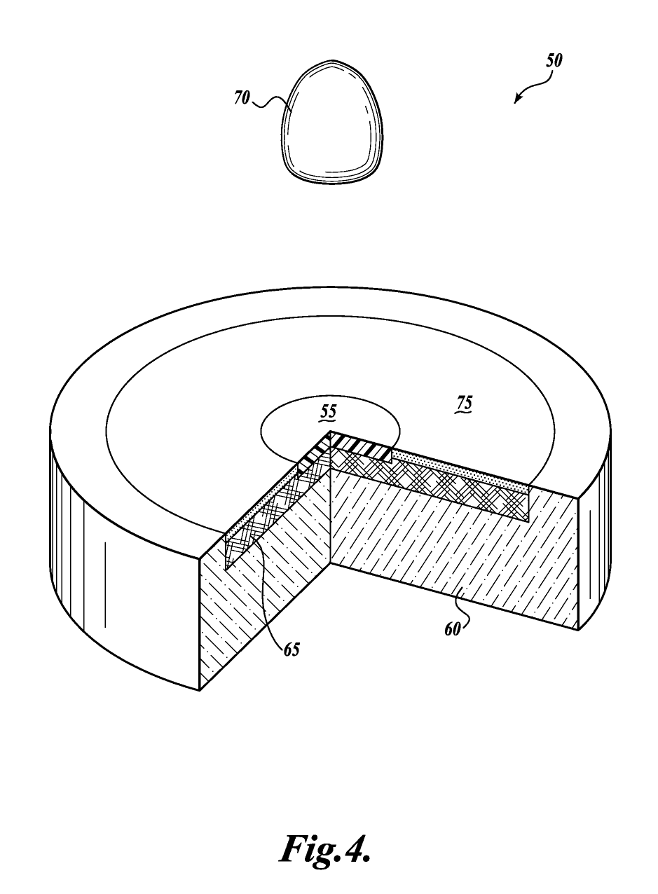

Referring to FIG. 4, another embodiment of the invention provides a flow-through device comprising a wicking system as a fluidic-transport means for moving a solution across a capture surface.

Referring to FIG. 4, a device is provided that includes a membrane, such as the membrane 15 described with reference to FIGS. 2, 3A, and 3B. The membrane is in intimate contact at a lower surface with an absorbent pad 65 configured to absorb a solution 70 by wicking the solution 70 through the membrane 55 and into the absorbent pad 65.

A heater 60 is provided on the device 50. The heater 60 is self-contained (e.g., a chemical heater).

The solution 70 comprises a plurality of aggregates (e.g., 150/250/350) such that the aggregates will be immobilized on a membrane 55 as the solution 70 passes through the membrane 55 into the absorbent pad 65.

In the illustrated embodiment of FIG. 4, a blocking material 75 is provided around the membrane 55 so as to contain the solution 70 within the surface area of the membrane 55. In this regard, the surface 75 is a material that will not transport the solution 70. For example, the surface 75 may be of opposite hydrophobicity as the solution 70. For example, if the solution 70 is hydrophilic, then the surface 75 is a hydrophobic material. In another embodiment, the surface 75 is impermeable to the solution 70, for example, a glass.

In operation of the device 50, the solution 70 is placed on the membrane 55, whereby it wicks through the membrane 55 into the wicking pad 65. The solution 70 is heated by the heater 60 above the LCST of the temperature-responsive polymer moieties in the aggregates 155 contained therein. The aggregates 155 are immobilized on the surface of the membrane 55 as the solution 70 passes through. Visual or other reporting techniques can be used to identify the presence of the aggregates 150 on the membrane after the solution 70 has completely passed through the membrane 55 and been absorbed into the pad 65.

### Visual Reporting Method

In another embodiment of the invention, a reporting moiety is incorporated into the aggregates so as to provide an easily identifiable (e.g., visual) indication of the presence of the immobilized aggregates (e.g., after filtering the aggregate solution).

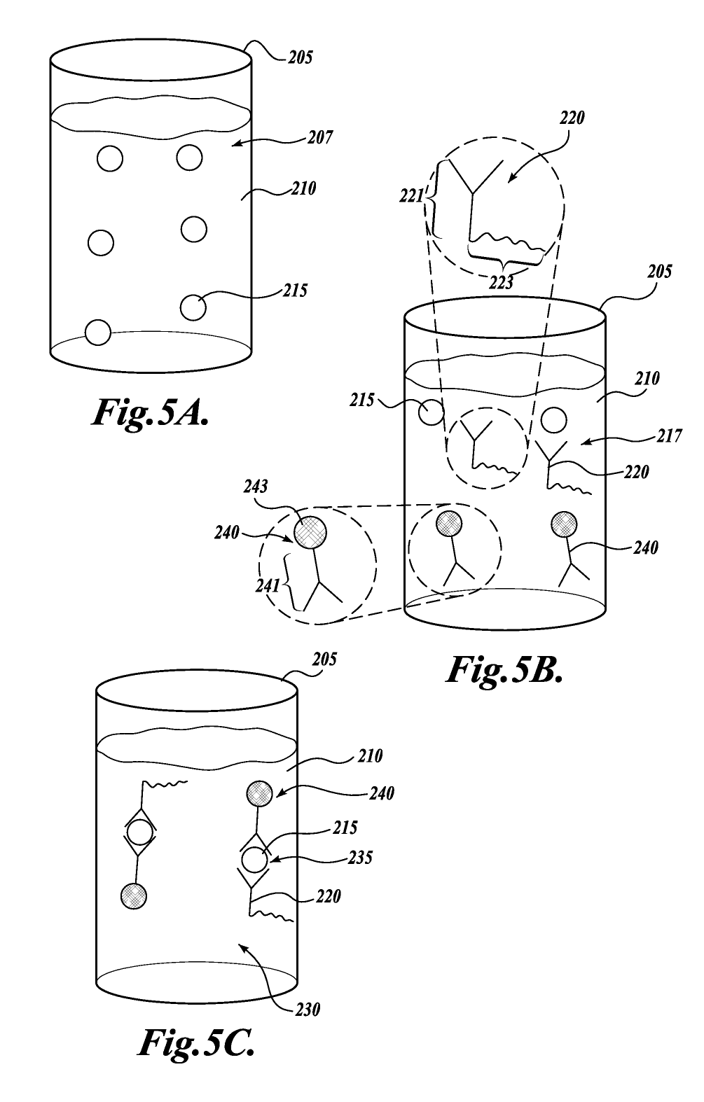

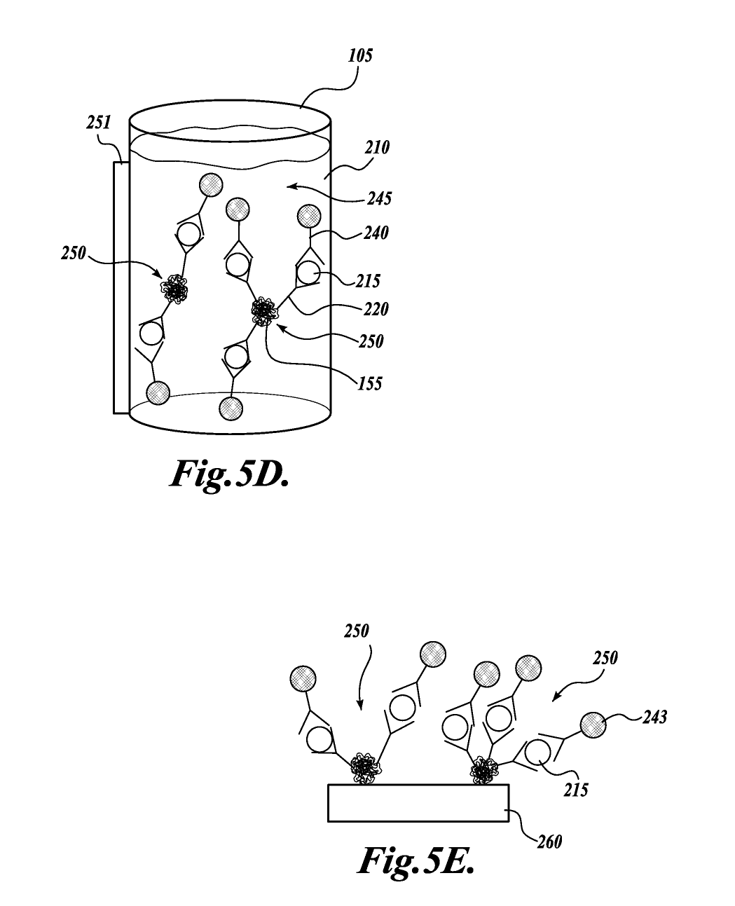

Referring to FIGS. 5A through 5E, a series of images similar to FIGS. 1A through 1E are presented. Similar to FIGS. 1A through 1E, the purpose of the steps illustrated in FIGS. 5A through 5E are to immobilize a diagnostic target 215 for identification. However, in the embodiments illustrated in FIGS. 5A through 5E, a reporting moiety (e.g., a visual indicator) is incorporated into the process.

Referring to FIG. 5A, a solution 207 containing a diagnostic target 215 in a biological fluid 210 is illustrated.

Referring to FIG. 5B, a solution 217 is provided comprising the biological fluid 210, the diagnostic target 115, a capture conjugate 220 comprising a first binding moiety 221 and a temperature-responsive polymer moiety 223, and a reporting conjugate comprising a second binding moiety 241 and a reporting moiety 243.

Regarding the reporting conjugate 240, the second binding moiety has a binding affinity to the diagnostic target 215 such that the second binding moiety 241 will bind to the diagnostic target 215 when in close proximity in solution. The second binding moiety can be any binding moiety capable of binding to the diagnostic target 215, similar to the first binding moiety 121/221 described above.

The reporting moiety 243 is a moiety configured to assist in reporting the presence of the diagnostic target 215. In one embodiment, the reporting moiety is selected from the group consisting of a metallic particle and a reporting enzyme. In one embodiment, the metallic particle is a gold particle. Gold particles are useful in visually identifying diagnostic targets 215 in the present invention because a sufficient concentration of gold particles will produce a color identifiable to human or mechanical vision so as to provide a simple, positive identification of a diagnostic target 115 attached to a gold particle.

Exemplary embodiments of the use of gold for identifying a diagnostic target are set forth below with regard to assays for HIV, malaria, and measles.

Reporting enzymes are also useful as a reporting moiety. The use of enzymes for visual identification is well known to those of skill in the art, such as in enzyme-linked immunosorbent assay (ELISA) techniques. If a reporting enzyme is the reporting moiety 243 on the reporting conjugate 240, the reporting enzyme can be later processed so as to contact a substrate to the enzyme, wherein the substrate produces a color change detectable by human or mechanical vision.

Referring to FIG. 5C, when in solution 230, the reporting conjugates 240 and capture conjugates 120 both bind to the diagnostic target 115 to form a capture complex 235. A plurality of capture complexes 235 can then be aggregated in a solution 245 having a temperature above the LCST of the temperature-responsive polymer moieties 223. Heat is provided by a self-contained heater 251, as illustrated in FIG. 5D. The aggregates 250 comprise a plurality of capture complexes 235, each capture complex comprising at least one diagnostic target and at least one reporting moiety 243.

Similar to the description above with reference to FIGS. 1D and 1E, the aggregates 250 are immobilized on the surface of the membrane 260. Due to the presence of the reporting moieties 243, each of which is attached to a diagnostic target 215, a visual indication of the presence of the diagnostic target in the initial solution 207 is provided upon immobilization on the membrane 260. That is, if a color appears on the membrane 260 after passing the solution 245 through the membrane 260, that color is definitively the result of a large number of aggregates 250, each containing at least one reporting moiety 243 and one diagnostic target 115. Therefore, the detectable color change can be positively stated as being attributable to the presence of the diagnostic target 115 in the solution.

It will be appreciated by those of skill in the art that additional processing steps may be required after the aggregates 250 are immobilized from the solution 245, such as illustrated in FIG. 5E. For example, additional reagents may be passed over the aggregates 250 so as to effect color change if an enzyme is used. Furthermore, the aggregates 250 may be removed from the membrane 260 via a liquid wash or other liquid-based concentration technique, and the aggregates 250 may be processed using other diagnostic methods or assays, such as lateral flow methods.

### Magnetic Particle Methods

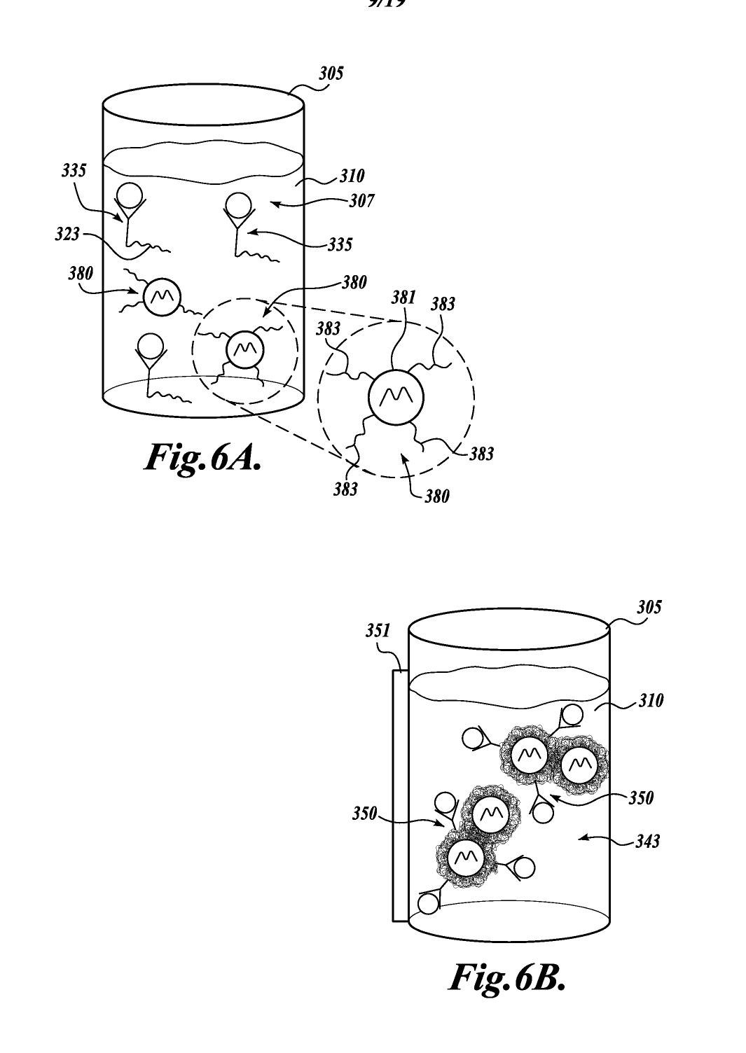

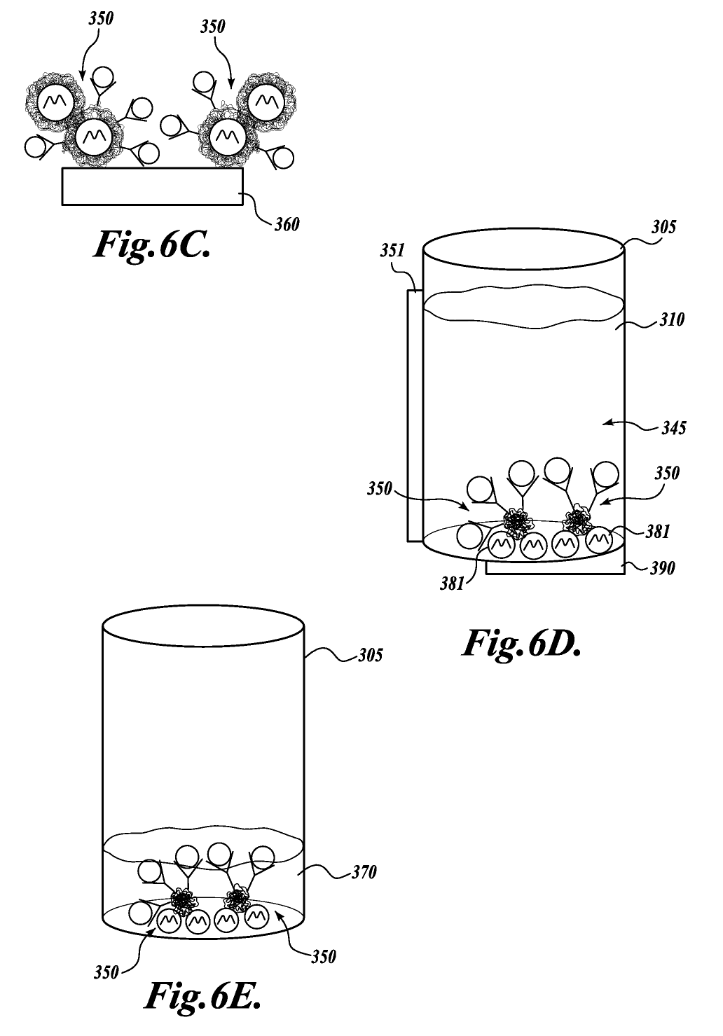

In further embodiments of the invention illustrated in FIGS. 6A through 6E, a system may be implemented whereby magnetic particles 380 are used to aggregate and isolate capture complexes 335. The capture complexes are similar to the capture complexes 135 or 235 described above. Referring to FIG. 6A, a solution 307 comprises capture complexes 335 and magnetic particles 380. The magnetic particles 380 each comprise a magnetic moiety 381 having one or more temperature-responsive polymer moieties 383 attached thereto.

Referring to FIG. 6B, the temperature of the solution 343 is raised, for example, by using a heater 351, above the LCST of the temperature-responsive polymer moieties 383 and 323, the magnetic particles 380, and the capture complexes 335 are aggregated together in the solution to form co-aggregates 350.

As illustrated in FIG. 6D, a magnet 390 can be used to immobilize the co-aggregates 350 in a magnetic field so as to concentrate the co-aggregates 350 in a particular portion of a container 305 comprising a solution 345 of the biological fluid 310 and the co-aggregates 350. Then, using techniques known to those of skill in the art, the supernatant of the solution 345 above the liquid level of the co-aggregates 350 can be removed to provide a concentrated solution 370 that contains all of the co-aggregates 350 previously in the larger volume of the solution 345. By increasing the concentration of the co-aggregates 350, the concentrated solution 370 can then be further processed, for example, by lateral flow methods to provide a stronger signal for detection of the diagnostic target 315 compared to a more dilute solution without co-aggregation and isolation.

Such magnetic techniques for isolating and immobilizing diagnostic targets 115 from a solution are the subject of U.S. patent application Ser. No. 12/815,217 filed Jun. 14, 2010 (“System and Method for Magnetically Concentrating and Detecting Biomarkers”), which is incorporated herein by reference in its entirety.

Alternatively, as set forth above with regard to FIGS. 1A through 1E and 2A through 2E, the co-aggregates 350 can be immobilized on a membrane 360 by filtration.

Both capture complexes with (not illustrated) and without reporting moieties are useful in the provided embodiments. That is, a reporting moiety can optionally be bound to the diagnostic target so as to provide a visual indication of captured diagnostic targets.

In another aspect of the invention, methods and systems are provided for forming aggregates comprising a magnetic particle and a capture conjugate. In certain embodiments, with reference to FIG. 6A, magnetic particles 380 are used to aggregate and isolate capture complexes 335. The capture complexes 335 are similar to the capture complexes 135 or 235 described above. The capture complexes comprise a first binding moiety, optionally bound to a diagnostic target, and a stimuli-responsive polymer moiety. Referring to FIG. 6A, a representative solution 307 comprises a biological fluid 310, capture complexes 335 (formed only when the diagnostic target is in the solution), and magnetic particles 380. The magnetic particles 380 each comprise a magnetic moiety 381 having one or more stimuli-responsive polymer moieties 383 attached thereto. In one embodiment, the stimuli-responsive polymer moieties on both the capture complex 335 and the magnetic particles 380 are pH-responsive and/or temperature-responsive polymer moieties. In one embodiment, the stimuli-responsive polymer moieties on both the capture complex 335 and the magnetic particles 380 are the same stimuli-responsive polymer moiety.

In FIG. 6A, the stimuli-responsive polymer moieties are in a non-associative state. Referring to FIG. 6B, a stimulus is applied to the solution 343 so as to initiate associative binding between the stimuli-responsive polymer moieties. For example, if the stimuli-responsive polymer moieties are pH-responsive polymer moieties, a buffer can be added to the solution 343 to change the pH of the solution to a pH value wherein the pH-responsive polymer moieties become associative to form co-aggregates 350. Alternatively, heat can be used in conjunction with temperature-responsive polymer moieties.

The presently-described aspect of the invention does not rely on heating, and particularly does not rely on self-contained heating to produce co-aggregates 350.

Once the co-aggregates 350 are formed they can be immobilized, isolated, concentrated, and/or interrogated using techniques known to those of skill in the art. For example, the co-aggregates 350 can be immobilized by subjecting them to a magnetic field. Once immobilized, the co-aggregates 350 can be interrogated to determine the presence of the diagnostic target.

In one embodiment, the magnetic particles 380 are magnetic nanoparticles. In one embodiment, the magnetic nanoparticles have a largest dimension of from about 5 nanometers to about 100 nanometers. Magnetic nanoparticles improve the kinetics of forming co-aggregates 350 compared to a system using micro, or larger, magnetic particles. The magnetic nanoparticles enable separation/enrichment of the diagnostic target bound to the magnetic nanoparticles when the aggregate size is large enough to achieve rapid magnetophoretic separations. This is unlike conventional magnetic enrichment schemes, where a magnetic particle is conjugated to a targeting ligand and forms one side of a “sandwich” immunocomplex”.

In one embodiment, the magnetic nanoparticles are paramagnetic magnetic nanoparticles. In one embodiment, the magnetic nanoparticles comprise iron oxide. In one embodiment, the magnetic nanoparticles are of a size and a composition such that a single magnetic nanoparticle will not effect magnetophoretic separation of a co-aggregate 350. Magnetophoretic separation is only effected using the magnetic nanoparticles when aggregated in co-aggregates 350 comprising a plurality of magnetic nanoparticles. The co-aggregates 350 of the invention, therefore, contain a plurality of magnetic nanoparticles, and a plurality of diagnostic targets. The plurality of magnetic nanoparticles in the co-aggregates 350 provides sufficient paramagnatism to enable magnetophoretic separation of the co-aggregates 350 in the solution 343.

After the co-aggregates 350 are formed in solution 343, a magnetic field is applied and the co-aggregates 350 are immobilized. Immobilized co-aggregates 350 can be concentrated (e.g., as illustrated in FIG. 6E) and/or washed with a series of solutions to identify any diagnostic target in the co-aggregates 350. Any technique know to those of skill in the art is useful for identifying the diagnostic target.

In one embodiment, an enzyme/substrate system is used whereby an enzyme is conjugated to a second binding moiety effective in recognizing the diagnostic target of the capture complex. The enzyme is then attached to the diagnostic target in the co-aggregates 350 via the second binding moiety. A substrate is then added to probe for the presence of the enzyme. A color change of the substrate indicates the presence of the diagnostic target.

### Magnetic Particle Device

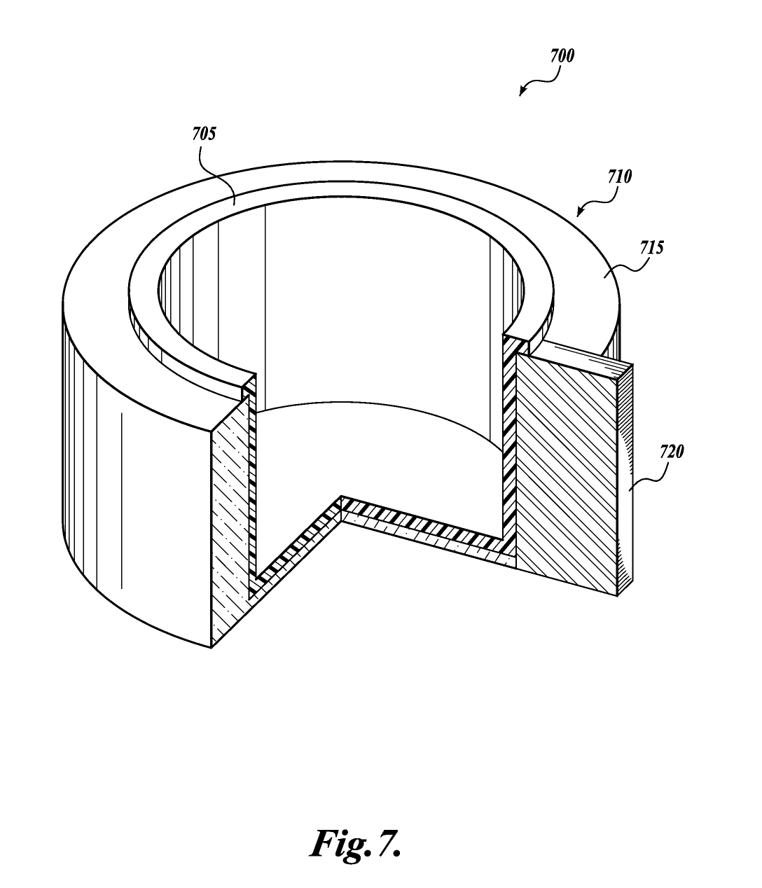

Referring to FIG. 7, an embodiment of a device useful for magnetically concentrating or immobilizing co-aggregates 350 such as those illustrated in FIGS. 6A through 6E, is provided. The magnetic device 700 comprises a solution container 705, nestingly fitted in a magnetic container 710 comprising a heater 715 and a magnet 720. In this embodiment, the capture surface is a region of the container affected by the magnetic field of the magnet.

The magnetic device 700 is useful, for example, for the method steps illustrated in FIGS. 6D and 6E, whereby co-aggregated particles comprising magnetic particles and capture complexes are formed through raising the temperature of the solution above the LCST of the temperature-responsive polymer moieties.

In the magnetic device 700, the heater 715 is a self-contained source of heat, as described elsewhere herein. Accordingly, the heater 715 of the magnetic device 700 is equivalent to the heater 351 in FIG. 6D. Relatedly, the magnet 720 of the magnetic device 700 is comparable to the magnet 390 illustrated in FIG. 6D. Therefore, when a solution (e.g., 345) is placed in the solution container 705 and heated by the heater 715 above the LCST, coaggregates (e.g., 350) are formed in the solution and are attracted to the magnet 720 such that they are immobilized and concentrated in the vicinity of the magnet 720.

As illustrated between FIGS. 6D and 6E, excess solution can be removed from the solution container so as to provide a solution with an increased concentration of co-aggregates 350. The co-aggregates 350 can then be removed in the concentrated solution and strip tested, or otherwise tested to determine the presence of diagnostic targets in the coaggregates.

Those of skill in the art will appreciate that the magnetic device 700 is only an exemplary embodiment of a magnet-containing device useful with the present invention. Magnets may be integrated into, for example, microfluidic devices or syringe-type devices, such as those illustrated in FIGS. 2, 3A, and 3B.

### PH-Responsive Polymers

While temperature-responsive polymers are used primarily to describe the methods and devices disclosed herein, pH-responsive polymers are also useful in certain embodiments of the invention. For example, any of the devices (e.g., FIGS. 2, 4, and 7) can be modified to function similarly using pH-responsive polymers. Or, alternatively, both pH and temperature responsivity can be used in a single method or device.

With regard to pH-responsive polymers substituted for temperature-sensitive polymers, a heater of the disclosed devices is not needed. Instead, a means for effecting pH change in the sample solution is needed. In one embodiment, the pH-modification means is a buffered solution miscible with the biological fluid. Such buffers are known to those of skill in the art. A modified device would exchange a heater for a means for providing a buffer of a predetermined pH.

Accordingly, in one another aspect, a device is provided for immobilizing a diagnostic target (e.g., antibody) from a solution. In one embodiment, the device comprises: a capture surface (e.g., a membrane) configured to immobilize an aggregate from a solution comprising a biological fluid and the aggregate, wherein the aggregate comprises a plurality of capture complexes each comprising the diagnostic target bound to a first binding moiety having a pH-responsive polymer moiety attached thereto, wherein the plurality of capture complexes are aggregated together through self-associative binding between the pH-responsive polymer moiety on each of the capture complexes; a pH-change means configured to change the pH of the solution to a predetermined pH value; and fluidic-transport means configured to move the solution across the capture surface.

Similarly, in another aspect, a method for concentrating a diagnostic target from a solution using a device is provided. In one embodiment, the device comprises a capture surface configured to immobilize an aggregate from a solution, a pH-change means configured to change the pH of the solution to a predetermined pH value, and a fluidic-transport means configured to move the solution across the capture surface, wherein the solution comprises a biological fluid and a capture complex comprising the diagnostic target bound to a capture conjugate comprising a pH-responsive polymer moiety bound to a first binding moiety (e.g., antibody) that has a binding affinity to the diagnostic target. The method for using the device includes the steps of: altering the pH of the solution to induce self-associative binding in the pH-responsive polymer moiety to provide an aggregate solution comprising the biological fluid and aggregates comprising a plurality of capture complexes aggregated through self-associative binding between the pH-responsive polymer moieties on each of the capture complexes; and flowing the aggregate solution past a capture surface configured to immobilize the aggregate, providing a captured aggregate.

Devices and methods that utilize both pH and temperature are provided in certain embodiments. The use of both pH and temperature can address a potential problem that may arise when using temperature-responsive polymers in warm climates. Particularly, because the heater of the present invention is self-contained, the temperature range over which it can heat is relatively small (e.g., 10 degrees C.). Therefore, the temperature-responsive polymer used in such a device is configured to be soluble at “ambient temperature” and insoluble (aggregated) at a temperature not more than 10 degrees above ambient temperature. Because “ambient temperature” is highly dependent on location, a test in the United States (25° C. ambient) may operate under very different conditions than one in Africa (35° C. ambient).

To address a disparity of potential temperatures, pH adjustments can be utilized in the invention to modify the polymer moieties on the conjugates so as to tune the LCST. For example, when using pNIAAm, the typical LCST is 32° C., meaning that the polymer will aggregate at an ambient temperature of 35° C. However, by using pNIAAm modified by a pH-responsive polymer (e.g., acrylic acid), a material is provided that has an adjusted LCST. In a representative embodiment, the temperature-responsive polymer is pNIAAm co-polymerized with an alkylacrylic acid (e.g., propylacrylic acid). Therefore, a warm-climate version of pNIAAm could be formulated that would have an LCST of, for example, 40° C. The same self-contained heater device disclosed herein could the be used to aggregate the polymer by raising the temperature from the ambient of 35° C., past the LCST of 40° C., to a maximum temperature of 45° C. for a length of time long enough to perform the aggregation and immobilization steps described elsewhere herein.

Similarly, the polymer can be engineered to aggregate in a particular pH range and a particular temperature range. For example, the polymer will aggregate only at pH ≦8.0 and temperature ≧40° C. Therefore, if the temperature is 38° C. and the pH is 7.4, the polymer conjugates do not aggregate. To aggregate the polymers, the temperature must be raised to ≧40° C., for example, by a self-contained heat source. Although, in this example, temperature is the only stimulus that drives the aggregation, the pH of the solution is still essential to the ability of the polymers to aggregate. That is, because the pH is below 8.0, aggregation is permitted by the pH-responsive polymer moieties. However, if the solution pH is >8.0, the polymer conjugates do not aggregate at the temperature ≧40° C. Therefore, it is the combination of pH and temperature that induces the aggregation. One advantage of this combination of pH and temperature control of aggregation is that the transition from clear solution to aggregation is very sharp because the aggregation mechanism includes both LCST and hydrogen bonding.

In another aspect, a device is provided that is configured to both heat the solution and to change the pH of the solution. Similarly, in another aspect, a method is provided that comprises the steps of adjusting the pH of the solution before and/or after heating the solution to produce aggregates.

HIV p24 protein Assay

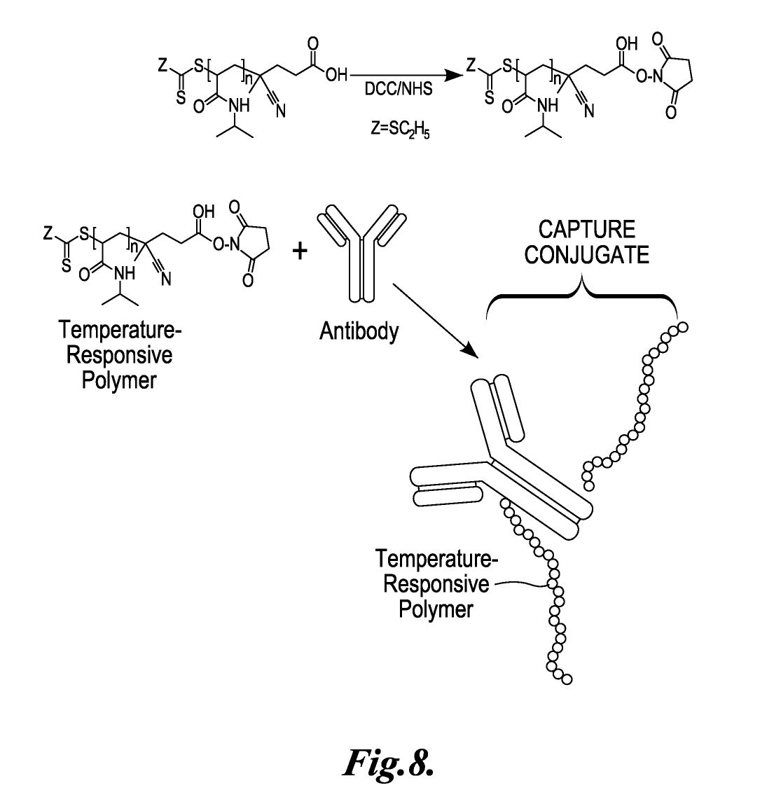

Exemplary devices and methods as disclosed herein were used to identify the presence of the p24 protein of HIV in human blood. As illustrated in FIG. 8, a capture conjugate was synthesized from an antibody and the temperature-responsive polymer PNIPAAm. Initially, the carboxylate chain end on the PNIPAAm polymer chain was “activated” using DCC/NHS. The “activated” polymer chains were then conjugated to the amine functional group on the antibody to form the capture complex having the antibody and a temperature-responsive polymer moiety. The PNIPAAm chains were synthesized using reversible addition-fragmentation chain transfer polymerization (RAFT) and contain a carboxylate chain end, which was used to covalently conjugate to the amine functional groups on the p24 antibodies via carbodiimide chemistry (e.g., DCC/NHS), as is known to those of skill in the art.

The carboxylate was activated (FIG. 8) in methylenechloride by mixing pNIPAAm:DCC:NHS at 1:1.1:1.1 ratio. The activation was allowed to proceed overnight at room temperature. The resulting activated polymer, NHS-pNIPAAm, was collected by precipitating in n-hexane. For conjugation, the NHS-pNIPAAm was pre-dissolved in anhydrous DMSO and added into p24 antibody solution (pH 8.5). The resulting reaction mixture contained 10% DMSO. The reaction was allowed to proceed overnight at 4° C. and then a desalting column was used to remove small molecule impurities. Capture conjugates, which exhibit temperature-responsiveness, were collected via centrifugation (10000 RPM, 5 minutes) at 40° C. The unmodified antibodies in the supernatant were discarded.

Capture conjugates were made using monoclonal p24 antibodies from commercially available sources, such as Maine Biotechnology Services (MBS), ImmunoDiagnostics, Inc. (IDI), and NIH. Different reaction stoichiometry (pNIPAAm:antibody molar ratio) was explored to achieve high conjugation efficiency and yield.

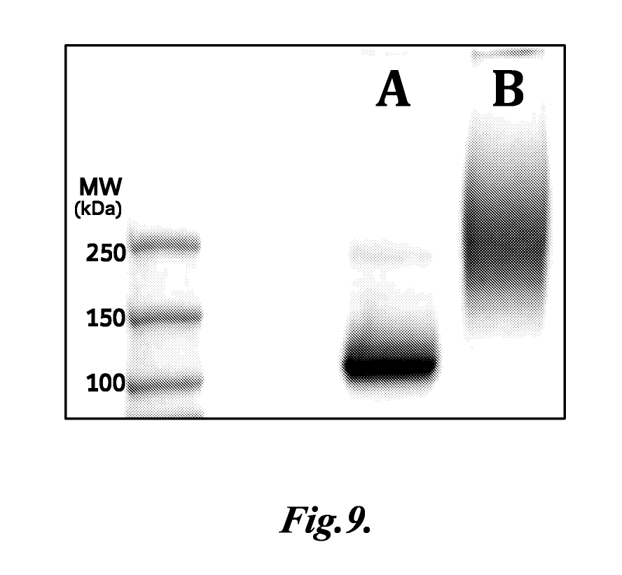

Sodium dodecyl sulfate polyacrylamide gel electrophoresis (SDS-PAGE) gel (FIG. 9) was used to confirm the polymer-antibody conjugation. Lane A is monoclonal p24 antibody supplied by MBS. Lane B is the pNIPAAm-antibody conjugate. The conjugate shows larger molecular weight than the native p24 antibody and Lane B shows no native p24 antibody band, which confirms complete conjugation.

The binding between the conjugates and p24 (antigen) was evaluated (and confirmed) using ELISA with human plasma samples spiked with p24. The conjugates were constructed by end-conjugating 30,000 molecular weight linear pNIPAAm polymer to monoclonal anti-p24 IgG. The conjugates were initially incubated with the human plasma samples spiked with p24 at room temperature for 10 minutes to establish binding between the conjugate and p24. The solution temperature was then raised to 40° C. for 15 minutes to induce anti-p24 conjugate aggregation. Afterward, the solution was centrifuged at 40° C. for 5 minutes to spin-down the conjugate aggregates with the bound p24. The supernatant was collected and analyzed for the amount of p24 using commercially available p24 ELISA. Antigen (p24)-conjugate binding results in the reduction of p24 in the collected supernatant. When the conjugate:p24 ratio increases from 16:1 to 16000:1, the p24 binding increases from ca. 40 to 90%. The binding reaches ca. 90%, when the conjugate:p24 ratio is ca. 1000:1.

Using the specification of heating to 40±3° C. target temperature for 30 minutes duration, an electricity-free heater device, such as that illustrated in FIGS. 2, 3A, and 3B, was fabricated. The engineering specifications for the exemplary device are set forth in Table 1.

TABLE 1
Specification of the prototype chemical heater developed for p24 assay.

| | |
|---|---|
| Functional temperature | 38 ± 5° C. |
| range | |
| Ambient temperature | 20-25° C. |
| range | |
| Ramp to functional | 5 minutes maximum |
| temperature | |
| Duration at functional | 15 minutes minimum |
| temp range | |
| Sample volume | 2 ml |
| Sample content | Plasma, DI water 50/50 mix |
| Sample reservoir | Basic BD 3 ml plastic syringe (e.g. P/N 309585). |
| geometry | |
| Process constraints | Before heating, user must be able to insert syringe |
| | assembly into heater. During heat step, user must |
| | be able to access and depress the syringe plunger. |
| | After heating, user must be able to remove |
| | assembly. |
| Heat Activation | Pouch design includes a reusable heater pack |
| | shaped as a syringe receptacle. Chemical heat is |
| | initiated by compressing a metallic button that |
| | produces an initial nucleation site for conversion |
| | of supersaturated sodium acetate liquid to a more |
| | stable crystalline state. This pouch can be |
| | disposable or can also be reused multiple times by |
| | recharging the sodium acetate in a heated water |
| | bath at a central facility or when electricity is |
| | available. |

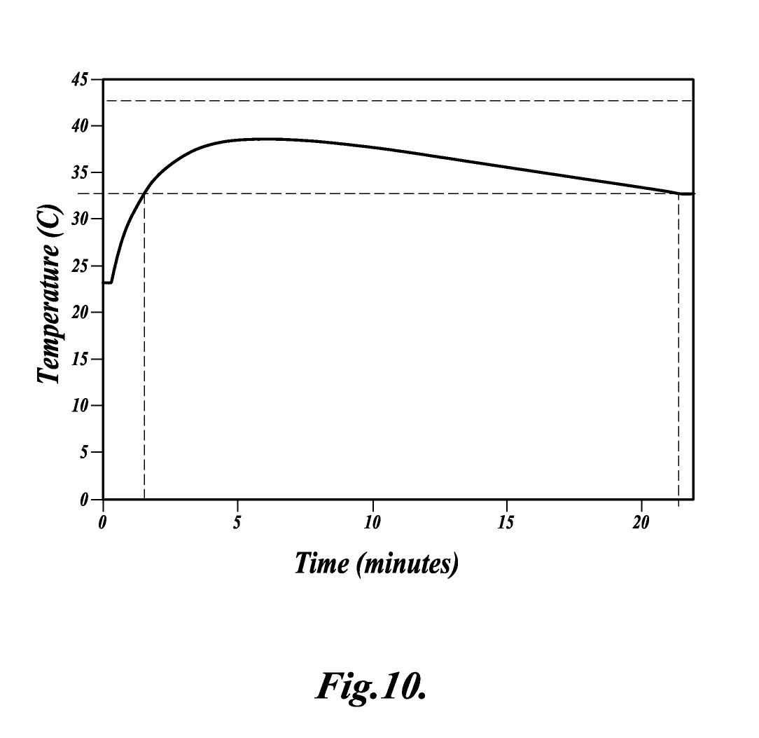

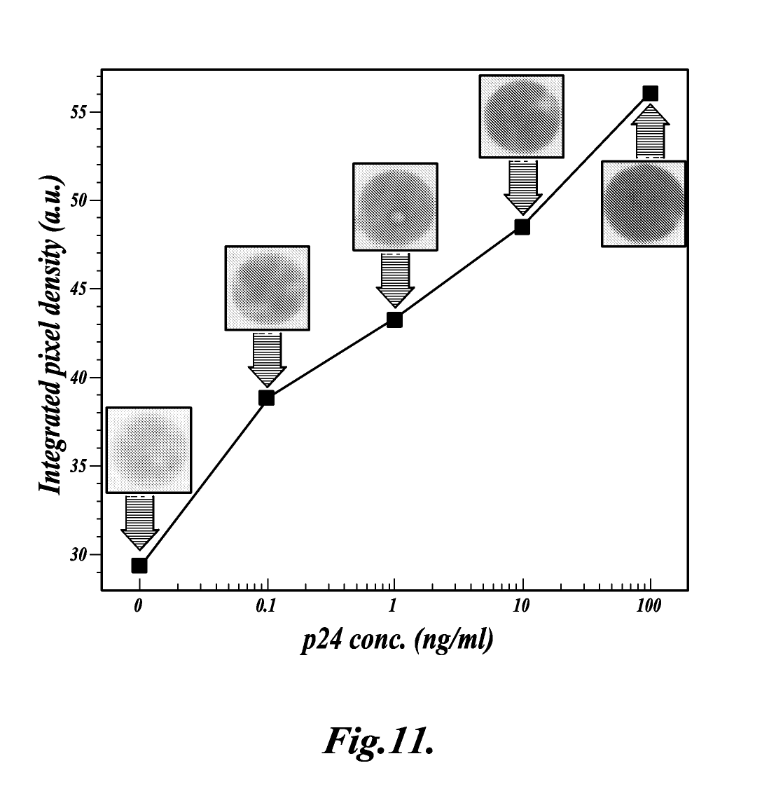

The heater was built with sodium acetate solution in a pouch and tested with thermocouples and a digital thermometer to assess performance (FIG. 10). The sodium acetate solution is 25% by weight in water. From the point of initiation, the heater reaches its peak temperature (ca. 40° C.) within 5 minutes and maintains above 32° C. for more than 20 minutes. This temperature change/duration is sufficient to drive a solution to above the LCST (ca. 32° C.) of PNIAAm.

The device was assembled using a 3 mL syringe. The membrane (1.2 micron pore size, LoProdyne® hydrophilic nylon) for immobilizing the aggregates is placed inside of a filter holder. The device was placed in the heater, to form a device similar to that illustrated in FIG. 2. The syringe plunger was removed before the assay was initiated. The sample solution, containing p24, anti-p24 capture conjugates, and anti-p24 gold reporting conjugates, was deposited into the syringe. Therefore, the solution in the syringe was similar to that illustrated in FIG. 5C.

Next, the heater was activated by initiating crystallization of the sodium acetate by providing a nucleation site by a metallic button. Once the solution was heated above the LCST, the plunger was placed into the syringe to move the sample fluid through the membrane. Therefore, the solution after heating was similar to that illustrated in FIG. 5D.

To complete the assay, all of the solution was moved through the membrane, which was retrieved and scanned to detect aggregates on the surface of the membrane similar to that illustrated in FIG. 5E. Membranes from filtering various concentrations of p24 were analyzed using a microscope and image analysis software. According to image analysis, illustrated in FIG. 8, the membranes for samples with p24 concentration from 0.1 to 100 ng/ml show signal higher than the background (0 ng/ml p24). As expected, the signal increases with increasing p24 concentration. In FIG. 8, the optical micrograph yielding the data for each point plotted on the graph is included.

Accordingly, p24 antibody was successfully isolated and visually identified using an exemplary device and method of the present invention.

### Malaria Assay

So as to test the methods and devices of the present invention for use as a malaria assay, an exemplary device was fabricated similar to the device described above with reference to the p24 assay (i.e., a device similar to that illustrated in FIG. 4).

A gold reporting moiety was utilized in this exemplary embodiment, and therefore, the process flow of testing for malaria, via the PFHRP2 antigen of malaria, from human plasma was carried out according to process steps as diagrammatically illustrated in FIGS. 5A through 5E. Aggregates were formed using PNIPAAm attached to a malaria antibody, which bound to the PFHRP2 antigen of malaria. Additionally in the solution was malaria antigen attached to a gold nanoparticle, as a reporting moiety. The three-part complexes were aggregated in solution above the LCST of the of the PNIPAAm via heat provided by a sodium acetate chemical heating pouch in contact with the chamber of the syringe holding the solution. Upon aggregation of the complexes, the solution was filtered through a 1.2 micron pore size hydrophilic nylon membrane. The aggregates were immobilized on the surface of the membrane while the remainder of the solution, including any non-bound components (e.g., non-bound reporting conjugates or capture conjugates) were allowed to pass through the membrane.

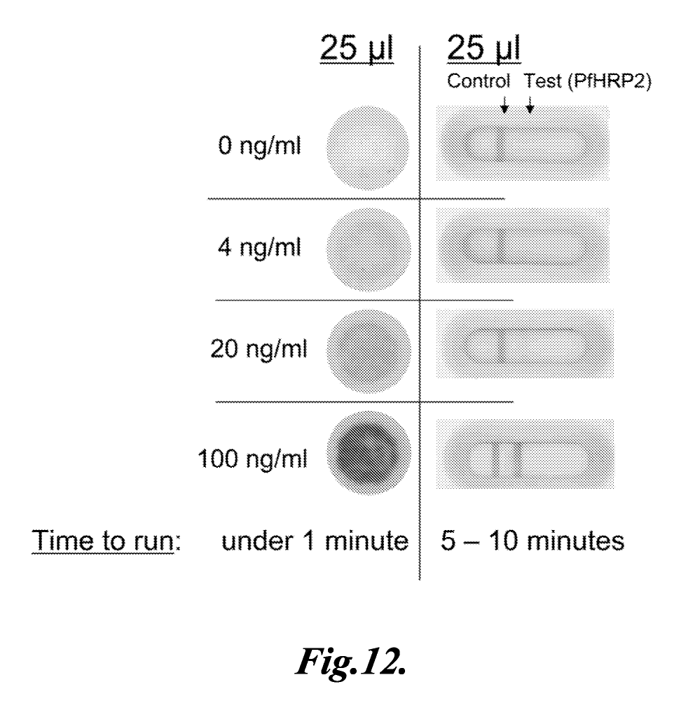

Referring to FIG. 12, in the left hand column, digital photographs provide a visual indication the presence of gold reporting moieties on the surface of the membrane. The sample size of solution pushed through the membrane was 25 microliters, and four different concentrations of PFHRP2 antigen were tested. At 0° ng/ml, no visual detection of PFHRP2 via gold is found. At 4 ng/ml, a faint circle is seen. At 20 ng/ml, a definitive circle is seen such that positive identification of PFHRP2 in the filtered solution can be made. At 100 ng/ml, the circle is dark and easily observable. Therefore, the limited detection in this exemplary embodiment would be between 4° ng/ml and 20 ng/ml of PFHRP2. The time to perform the assay is under one minute.

Still referring to FIG. 12, a control test utilizing 25 microliters of the same PFHRP2 human plasma solutions as described above was used. The control test was a commercially available Sanitoets MAL assay rapid flow test for PFHRP2. The rapid flow test is a visual indicator test. The rapid flow test takes from 5 to 10 minutes and, as can be seen from the images in the right hand column of FIG. 12, the rapid flow test does not detect PFHRP2 in the human plasma until a concentration of 100 ng/ml is reached.

Therefore, the device and method of the present invention is up to an order of magnitude faster in performing the PFHRP2 assay than the commercially available rapid flow test, and potentially an order of magnitude more sensitive so as to allow for diagnosis of malaria even with low concentrations of malaria antigen in a patient's blood.

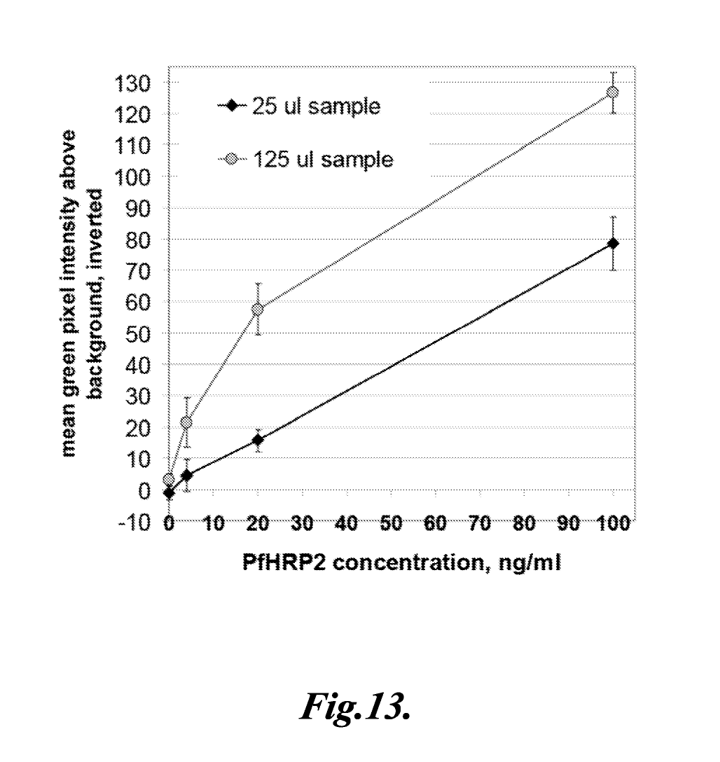

Referring to FIG. 13, a graph illustrates the relative sensitivity to PFHRP2 concentration of the exemplary device and methods described above with reference to FIG. 12. The pixel intensities measured for the images acquired through testing of the different concentrations of PFHRP2, as described above, are graphed in FIG. 13, in addition to a series of data representing the same test performed on five times the volume of human plasma (i.e., 125 microliters). As indicated in FIG. 13, the greater the volume of sample, the higher signal produced, and the easier visual diagnosis can be achieved using the present invention.

### Measles Assay

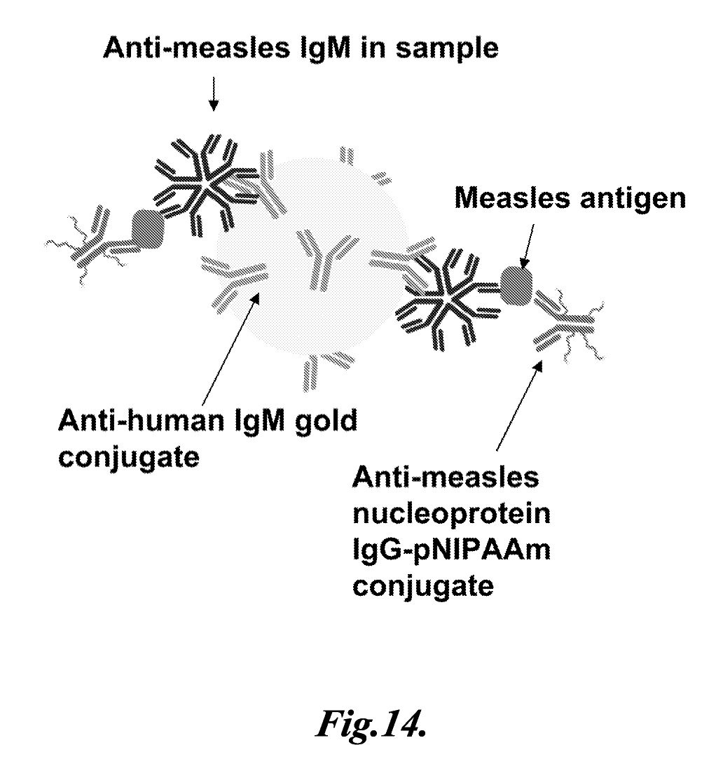

In addition to the assays discussed above with regard to p24 and malaria, serology may also be used as an aspect of the present invention. In this regard, FIG. 14 illustrates the use of the present invention with serology to diagnose measles. As illustrated in FIG. 14, measles is detected by assaying for the anti-measles IgM in a sample of human plasma. The anti-measles IgM is visually detectable using the present invention by providing anti-human IgM conjugated to a gold nanoparticle. The anti-human IgM binds specifically to the anti-measles IgM thus providing a gold nanoparticle tether conjugated to the anti-measles IgM. In order to immobilize and concentrate the anti-measles IgM bound to the anti-human IgM gold conjugate, a conjugate of measles antigen, anti-measles nucleoprotein IgG-PNIPAAm is also used in the solution. As illustrated in FIG. 14, the PNIPAAm conjugate binds specifically to the anti-measles IgM through the affinity of the measles antigen to the anti-measles IgM.

As described above with reference to the p24 and malaria assays, the anti-measles IgM is a diagnostic target that is bound in solution to a gold reporting moiety and a conjugate having PNIPAAm attached thereto. By raising the temperature of the solution above the LCST of the PNIPAAm, using a self-contained source of heat (a sodium acetate heating package), the PNIPAAm becomes self-associative and forms aggregates with other PNIPAAm conjugates in the solution. The aggregates are then captured, using a device similar to that illustrated in FIG. 4, on the surface of the membrane (as described above, via adhesive forces between the membrane and aggregates), which changes color as a result of the gold reporting moieties bound to the anti-measles IgM. Therefore, the color change on the surface of the membrane indicates concentration of anti-measles IgM in the sample.

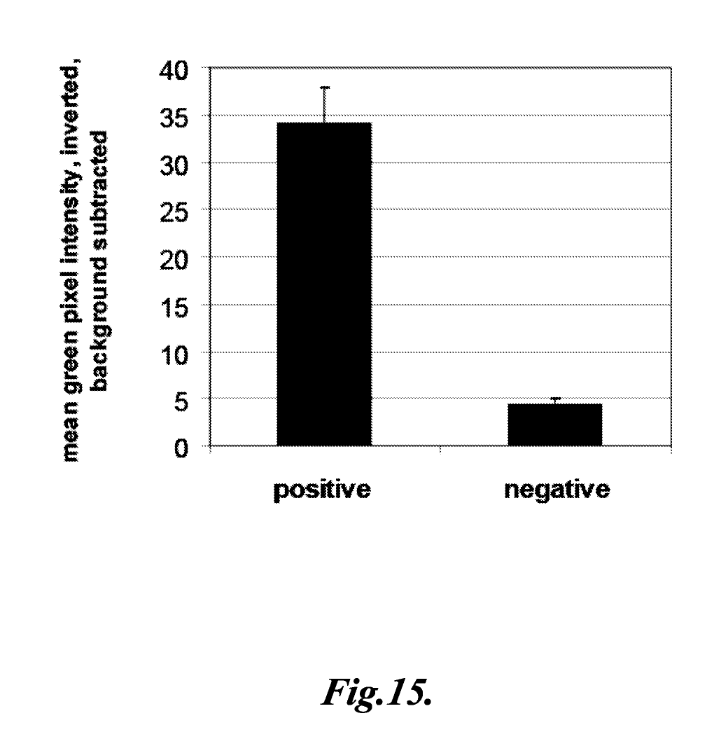

As illustrated in FIG. 15, graphically, the “positive” sample, which contains the complex illustrated in FIG. 14, produces a greater color change, measured by green pixel intensity, than the “negative” sample, which is normal human plasma without any gold reporting moieties or PNIPAAm conjugates in the plasma. Accordingly, these exemplary results indicate that serology can be used in the present invention to diagnose diseases in biological fluids, in the present case, diagnosing measles from human plasma via anti-measles IgM.

### Diagnostic Kit

The devices of the invention can be packaged into a diagnostic kit comprising the device and the necessary compounds to perform an assay for a selected diagnostic target. As discussed above, various combinations of capture conjugate, reporting conjugate, and magnetic particle can be used to perform the methods of the present invention. Therefore, a kit of the present invention includes at least the capture conjugate, and optionally includes the reporting conjugate and/or the magnetic particle. The conjugates/particles can be dried or solvated and packaged into the kit for easy use. For example, pre-apportioned amounts of the conjugates/particles can be provided with the device such that the conjugates/particles are added to the biological fluid held in the device so as to capture, aggregate, and immobilize the diagnostic target.

### Multiple-Diagnostic Targets

While the embodiments disclosed herein have been described with reference to a single diagnostic target, it will be appreciated that the methods can be modified to test for multiple diagnostic targets. Similarly, the devices can be modified to perform multiple capture/reporting cycles so as to report on the presence of multiple diagnostic targets.

### Temperature-Responsive Polymer Membranes

In certain embodiments, the devices and methods of the present invention utilize a membrane (e.g., part 15 of FIG. 2). As described above, a temperature-responsive polymer membrane can be used to increase the adhesion between aggregates and the membrane to improve immobilization efficiency. An exemplary embodiment is discussed below regarding forming a membrane including temperature-responsive polymers. Experimental conditions and results are included.

Uniform coverage of the membrane with narrow molecular weight distribution temperature-responsive polymer is desired. The membrane modification therefore combines a “graft-from” technique together with RAFT polymerization to control the membrane functional properties. Hydroxylated nylon membranes contain activated hydroxyl groups on the surface, so the RAFT CTA (2-ethylsulfanylthiocarbonylsulfanyl-2-methyl propionic acid) can be immobilized on the membrane via the end carboxyl group using carbodiimide chemistry as discussed previously herein. The surface coverage can be adjusted by varying the CTA concentration. The reaction is carried out for 48 hours at room temperature and membranes are then extensively washed in acetone and ethanol alternatingly, and then followed by washing in distilled water. After drying by vacuum at room temperature, the membrane is then stored under ambient conditions.

Polymerization on the membrane is mediated by the grafted CTA using RAFT polymerization. Standard solution polymerization conditions are followed and membranes with bound CTA are included in the solution during the polymerization. NIPAAm concentration is at 0.4 g/mL with AIBN as initiator. Polymerization is performed at 60° C. under nitrogen for 18 hours. Solution polymer is retained and analyzed. The membranes are washed extensively with ethanol and soaked at 4° C. for 48 hours or longer in several changes of distilled water to remove non-covalently adsorbed or entangled polymers.

The membrane modification is evaluated by determining the molecular weight and the polydispersity index of the grafted PNIPAAm. The grafted PNIPAAm can be cleaved by treating the membranes with 1N NaOH (approximately 2 mL per cm² of membrane) and heating at 70° C. for 1 hour to hydrolyze the ester linkage between the polymer and the membrane. The collected solutions are neutralized with 1N HCl and dialyzed against distilled water for 48 hours. Dialyzed solutions are then lyophilized and characterize using SEC, which confirmed the presence of PNIPAAm.

While illustrative embodiments have been illustrated and described, it will be appreciated that various changes can be made therein without departing from the spirit and scope of the invention.

The embodiments of the invention in which an exclusive property or privilege is claimed are defined as follows:

1. A device for immobilizing a diagnostic target from a solution, comprising:
a capture surface configured to immobilize an aggregate from a solution comprising a biological fluid and the aggregate, wherein the aggregate comprises a plurality of capture complexes each comprising the diagnostic target bound to a first binding moiety having a temperature-responsive polymer moiety attached thereto, wherein the plurality of capture complexes are aggregated together through self-associative binding between the temperature-responsive polymer moiety on each of the capture complexes;
a self-contained source of heat configured to deliver a predetermined amount of heat for a predetermined amount of time to the solution, wherein the predetermined amount of heat is sufficient to raise the temperature of the solution above a lower critical solution temperature (LCST) of the temperature-responsive polymer moiety for the predetermined amount of time; and
fluidic-transport means configured to move the solution across the capture surface.

2. The device of claim 1, wherein the capture surface is a planar membrane having an inlet surface opposite an outlet surface.

3. The device of claim 2, wherein the membrane is configured to immobilize the diagnostic target through a binding mechanism selected from the group consisting of hydrophilic-hydrophilic affinity, hydrophobic-hydrophobic affinity, hydrogen bonding, and self-associative affinity binding.

4. The device of claim 3, wherein the fluidic-transport means is a wicking system comprising an absorbent pad abutting the outlet surface of the membrane, wherein the wicking system is configured to move the solution in contact with the inlet surface of the membrane through the membrane to the outlet surface and into the absorbent pad.

5. The device of claim 3, wherein the fluidic-transport means is a forced-flow system configured to move the solution through the membrane using pressure applied to the solution.

6. The device of claim 5, wherein the forced-flow system is a syringe system comprising a container in fluid communication with the inlet surface of the membrane, wherein the container is configured to hold the solution, and wherein the container comprises a plunger configured to apply pressure to the solution in the container such that the solution is forced into contact with the membrane at the inlet surface.

7. The device of claim 2, wherein the membrane comprises the temperature-responsive polymer moiety.

8. The device of claim 2, wherein the capture complex further comprises a reporting conjugate comprising a reporting moiety bound to a second binding moiety, wherein the second binding moiety is bound to the diagnostic target.

9. The device of claim 8, wherein the reporting moiety is a visual reporting moiety selected from the group consisting of a gold particle and a reporting enzyme.

10. The device of claim 1, wherein the capture surface is within a magnetic field, wherein the magnetic field is configured to immobilize a co-aggregate from the solution, wherein the co-aggregate comprises the aggregate and a magnetic particle comprising a magnetic moiety bound to the temperature-responsive polymer moiety, wherein the co-aggregate is aggregated through self-associative binding between the temperature-responsive polymer moieties on the capture complexes of the aggregate and on the magnetic particles.

11. The device of claim 10 further comprising a container in fluid communication with the capture surface.

12. The device of claim 11, wherein the capture surface is within the container.

13. The device of claim 10, wherein the magnetic field is generated by a permanent magnet.

14. The device of claim 1, wherein the biological fluid is selected from the group consisting of blood, mucus, urine, tissue, sputum, saliva, feces, a nasal swab, and nasopharyngeal washes.

15. The device of claim 1, wherein the diagnostic target is an antibody or antigen for a disease selected from the group consisting of human immunodeficiency virus, malaria, dengue, salmonella, rickettsia, influenza, chlamydia, prostate cancer and measles.

16. The device of claim 1, wherein the diagnostic target is selected from the group consisting of a p24 protein of human immunodeficiency virus, a PfHRP2 antigen of malaria, an aldolase antigen of malaria, NS1 antigen of dengue, flagella/somatic/Vi antigens of salmonella, nucleoprotein/hemagglutinin antigens of influenza, LPS antigen of Chlamydia, prostate-specific antigen of prostate cancer, and antibodies of diseases selected from the group consisting of dengue, salmonella, and rickettsia.

17. The device of claim 1, wherein the self-contained source of heat is a non-electric source of heat.

18. The device of claim 1, wherein the self-contained source of heat is a phase-change material.

19. The device of claim 1 further comprising a container in fluid communication with the capture surface, wherein the self-contained source of heat abuts the container.

20. The device of claim 1, wherein the capture surface, the self-contained source of heat, and the fluidic-transport means are all contained in a hand-held package.

21. The device of claim 1, wherein the temperature-responsive polymer moiety is a derived from a monomer selected from the group consisting of N-isopropylacrylamide, tert-butyl methacrylate, tert-butyl acrylate, butyl methacrylate, butylacrylate, dimethylaminoethyl acrylamide, and propylacrylic acid.

22. The device of claim 1, wherein the temperature-responsive polymer moiety comprises a pH-responsive polymer moiety.

23. A method for concentrating a diagnostic target from a solution using a device comprising a capture surface configured to immobilize an aggregate from a solution, a self-contained source of heat configured to deliver a predetermined amount of heat for a predetermined amount of time to the solution, and a fluidic-transport means configured to move the solution across the capture surface, wherein the solution comprises a biological fluid and a capture complex comprising the diagnostic target bound to a capture conjugate comprising a temperature-responsive polymer moiety bound to a first binding moiety that has a binding affinity to the diagnostic target, the method comprising:
heating the solution with the self-contained source of heat to a temperature above a lower critical solution temperature (LCST) of the temperature-responsive polymer moiety to provide an aggregate solution comprising the biological fluid and aggregates comprising a plurality of capture complexes aggregated through self-associative binding between the temperature-responsive polymer moieties on each of the capture complexes; and
flowing the aggregate solution past a capture surface configured to immobilize the aggregate, providing a captured aggregate.
